(19) United States
(12) Patent Application Publication
(10) Pub. No.: US 2018/0022810 A1
(43) Pub. Date: Jan. 25, 2018

(54) USE OF CXCR3 INHIBITORS FOR PROTECTING AGAINST FETAL WASTAGE

(71) Applicant: Children's Hospital Medical Center, Cincinnati, OH (US)

(72) Inventor: Sing Sing Way, Cincinnati, OH (US)

(73) Assignee: Children's Hospital Medical Center, Cincinnati, OH (US)

(21) Appl. No.: 15/548,814

(22) PCT Filed: Feb. 4, 2016
(86) PCT No.: PCT/US16/16533

§ 371 Date: Aug. 4, 2017

Related U.S. Application Data
(60) Provisional application No. 62/112,529, filed on Feb. 5, 2015.

(51) Int. Cl. C07K 16/28

(52) U.S. Cl. CPC C07K 16/2866; C07K 2317/76; A61K 2039/505

(57) ABSTRACT

Provided herein are compositions and methods related to reducing the risk of or preventing fetal wastage in a subject using a CXCR3 inhibitor.

### CROSS-REFERENCE TO RELATED APPLICATIONS

This application claims the benefit under 35 U.S.C. §119(e) of U.S. provisional application No. 62/112,529 filed Feb. 5, 2015, the contents of which are incorporated by reference herein in their entirety.

### BACKGROUND OF INVENTION

Stillbirth remains a pressing global health problem, with an estimated 2.6 million cases occurring annually. It is suggested that maternal infection is an important causative factor in stillbirth. The heterogeneity of microbes causing maternal infection and the rapidity that these infections can cause fetal injury preclude the use of antimicrobials targeting individual pathogens as a means of therapy or prevention.

As a result, there exists a need for developing new approaches to treat or prevent fetal injury caused by prenatal infection.

### SUMMARY OF THE INVENTION

The present disclosure is based on the unexpected discovery that neutralizing the CXCR3 receptor (e.g., by an antibody that neutralizing the activity of CXCR3) successfully inhibited fetal-specific CD8⁺ T cells in a pregnant subject and prevented fetal wastage under both infection and non-infection context.

Accordingly, described herein are methods and compositions related to reducing the risk of or preventing fetal wastage in a pregnant subject by administering a CXCR3 inhibitor.

Accordingly, aspects of the disclosure relate to use of a CXCR3 inhibitor to reduce the risk of or prevent fetal wastage in a pregnant subject, such as a subject having an infection or at risk of having an infection.

In some aspects, the disclosure relates to a method of reducing the risk of or preventing fetal wastage, the method comprising administering to a pregnant subject (e.g., a human female subject) a CXCR3 (e.g., human CXCR3) inhibitor in an amount effective to reduce the risk of or prevent fetal wastage. In some embodiments, the amount of the CXCR3 inhibitor is effective to inhibit fetal-specific CD8⁺ T cells in the subject.

In some embodiments, the subject has or is at risk of having an infection, a stillbirth, preeclampsia or a premature infant. In one example, the subject has or is at risk of having an infection, which may be caused by a pathogen. Exemplary pathogens include, but are not limited to Listeria monocytogenes, Influenza A, herpes simplex virus, Lymphocytic Choriomeningitis Virus (LCMV), Salmonella, Plasmodium, Toxoplasma, Escherichia coli, CMV (cytomegalovirus), parvovirus, or Leishmania spp. In one example, the infection is caused by Listeria monocytogenes.

The CXCR3 inhibitor may be administered prior to the infection (e.g., an infection caused by Listeria monocytogenes) or prior to manifestation of a symptom of the infection. For example, the CXCR3 inhibitor may be administered to the subject within 7 days after the subject is infected with the pathogen or manifests a symptom of the infection.

In any of the methods described herein, the CXCR3 inhibitor can be an interfering RNA, an antisense oligonucleotide, a small molecule, and an antibody. In some embodiments, the CXCR3 inhibitor is an antibody that specifically binds to CXCR3 and neutralizes CXCR3 activity. In some examples, the antibody can be a human antibody or a humanized antibody

Also within the scope of the present disclosure are (a) a pharmaceutical composition for use in reducing the risk or preventing fetal wastage in a pregnant subject, the composition comprising any of the CXCR3 inhibitors described herein and a pharmaceutically acceptable carrier; and (b) use of such CXCR3 inhibitor in manufacturing a medicament for use in reducing the risk or preventing wastage in a pregnant subject.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 consists of graphs showing that maternal CD8⁺ T cells are essential for prenatal Lm infection induced fetal wastage. (A) Percent fetal resorption and number of live pups five days after Lm ΔactA (10⁷ CFU) infection initiated midgestation (E11.5) among C57BL/6 wildtype (WT) compared with Rag2-deficient female mice bearing allogeneic pregnancy sired by Balb/c males and no infection controls. (B) Percent fetal resorption and number of live pups five days after Lm ΔactA (10⁷ CFU) infection initiated midgestation (E11.5) among C57BL/6 female mice bearing allogeneic pregnancy sired by Balb/c males treated with anti-CD4 and/or anti-CD8, or anti-IFN-γ antibody each compared with rat IgG control antibody (500 μg/mouse) one day prior to infection and no infection controls. Each symbol reflects the data from a single mouse, and these data are representative of at least three independent experiments each with similar results. Bar, mean±one standard error. Statistical analysis between groups was evaluated using an unpaired Student's t test.

FIG. 2 shows that prenatal infection induced fetal resorption requires maternal CD8⁺ T cells with fetal specificity. (A) Percent fetal resorption and number of live pups five days after Lm ΔactA or Lm ΔactA OVA infection (each 10⁷ CFU) initiated midgestation (E11.5) among P14 or OT-1 TCR transgenic mice during allogeneic pregnancy sired by Balb/c or Balb/c-OVA males. Ten days before mating, P14 and OT-1 TCR transgenic mice maintained on a Rag2-deficient background were reconstituted with polyclonal CD4⁺ T and B cells from splenocytes of CD8α-deficient mice. (B) Representative FACS plots and composite data showing percent IFN-γ production after PMA/ionomycin stimulation among maternal CD8⁺ splenocytes recovered five days after Lm ΔactA or Lm ΔactA OVA infection (each 10⁷ CFU) for the mice described in panel A. Each symbol reflects the data from a single mouse, and these data are representative of at least three independent experiments each with similar results. Bar, mean±one standard error.

FIG. 3 shows the placental accumulation of maternal CD8⁺ T cells with fetal specificity is triggered by prenatal Lm infection. (A) Representative FACS plots and composite data showing percent fetal-OVA₂₅₇₋₂₆₄-specific (CD90.1⁺) among CD8⁺ T cells recovered from the decidua three days after Lm ΔactA (10⁷ CFU) infection initiated midgestation (E11.5) among C57BL/6 female mice during allogeneic pregnancy sired by Balb/c-OVA compared with non-transgenic Balb/c males and no infection controls. Each symbol reflects the data from a single mouse, and these data are representative of at least three independent experiments each with similar results. (B) Representative histological analysis of the placenta recovered from mice described in panel A showing no infection control (top) compared with Lm ΔactA (10⁷ CFU) infection (bottom) among OVA⁺ concepti after hematoxylin and eosin (H&E), along with anti-CD90.1 (red) and 4′,6-diamidino-2-phenylindole (DAPI) nuclear immunofluorescence staining. High (100×) magnification fields show placental tissue intersecting the decidua basalis (DB) and junctional zone (JZ). Brackets in the low (4×) magnification fields indicate source of decidual tissue harvested for analysis by flow cytometry. These data are representative of >10 individual placenta from at least three separate litters for each experimental group. Bar, mean±one standard error. Myo, myometrium; DB, decidua basalis; JZ; junctional zone; Lab, labyrinth; CP, chorionic plate.

FIG. 4 shows that CXCL9 producing inflammatory cells accumulate in the decidua after prenatal Lm infection. (A) Number of CD45⁺ leukocyte and CD45⁻ non-leukocyte stromal cells recovered from the decidua each time point after Lm ΔactA (10⁷ CFU) infection initiated midgestation (E11.5) among C57BL/6 female mice during allogeneic pregnancy sired by Balb/c males. (B) Pie chart illustrating quantitative accumulation and quantitative shifts in each CD45⁺ leukocyte subsets recovered from the decidua for mice described in panel A. Individual leukocyte subsets were delineated after gating on CD45⁺ cells, and identified as neutrophils (CD11b⁺ Ly6C^{int}); macrophages (F4/80⁺ CD11b⁻); natural killer cells (NK1.1⁺ CD4⁻ CD8⁻); B cells (B220⁺ CD4⁻ CD8⁻); CD4 cells (CD4⁺ CD8⁻); and CD8 cells (CD8⁺ CD4⁻). (C) Relative CXCL9 expression among cells recovered from the decidua compared with adjacent myometrium after Lm ΔactA (10⁷ CFU) infection for mice described in panel A. (D) Relative CXCL9 expression among CD45⁺ compared with CD45⁻ decidual cells, and representative histogram plots showing CXCL9 expression by each cell type before (gray shaded), and 24 (blue line) or 72 (red line) hours after Lm ΔactA infection. Each symbol reflects the data from a single mouse, and these data are representative of at least three independent experiments each with similar results. Bar, mean±one standard error.

FIG. 5 shows that the depletion of CXCL9 producing neutrophil and macrophage cells protects against Lm infection induced fetal wastage. (A) Percent fetal resorption five days after Lm ΔactA (10⁷ CFU) infection initiated midgestation (E11.5) among C57BL/6 female mice bearing allogeneic pregnancy sired by Balb/c males administered anti-Gr1 compared with isotype control antibody (500 μg/mouse) one day prior to infection. (B) Representative FACS plots and composite data showing percent fetal-OVA₂₅₇₋₂₆₄ specific (CD90.1⁺) among CD8⁺ T cells recovered from the decidua three days after Lm ΔactA (10⁷ CFU) infection for C57BL/6 female mice bearing allogeneic pregnancy sired by Balb/c-OVA males administered anti-Gr1 compared with isotype control antibody (500 μg/mouse) one day prior to infection. Each symbol reflects the data from a single mouse, and these data are representative of at least three independent experiments each with similar results. Bar, mean±one standard error. Statistical analysis between groups was evaluated using an unpaired Student's t test.

FIG. 6 shows that prenatal Lm infection selectively primes CXCR3 expression by maternal CD8⁺ T cell with fetal specificity. Representative plots and composite analysis showing relative expression of CXCR3 by OVA₂₅₇₋₂₆₄-specific (CD90.1⁺) CD8⁺ T cells recovered from the decidua or spleen among C57BL/6 female mice bearing allogeneic pregnancy sired by Balb/c-OVA compared with Balb/c males three days after Lm ΔactA (10⁷ CFU) infection initiated midgestation (E11.5) and no infection controls. Each symbol reflects the data from a single mouse, and these data are representative of at least three independent experiments each with similar results. Bar, mean±one standard error. Statistical analysis between groups was evaluated using an unpaired Student's t test.

FIG. 7 shows that CXCR3 deprivation protects against prenatal Lm infection induced fetal wastage. (A) Percent fetal resorption among C57BL/6 compared with isogenic CXCR3-deficient female mice five days after Lm ΔactA (10⁷ CFU) infection initiated midgestation (E11.5) during allogeneic pregnancy sired by Balb/c males and no infection controls. (B) Percent fetal resorption among C57BL/6 female mice five days after Lm ΔactA (10⁷ CFU) infection initiated midgestation (E11.5) among C57BL/6 female mice during allogeneic pregnancy sired by Balb/c males administered anti-CXCR3 compared with isotype control antibody (500 μg/mouse) one day prior to infection and no infection controls. (C) Representative FACS plots and composite data showing percent fetal-OVA₂₅₇₋₂₆₄ specific (CD90.1⁺) among CD8⁺ T cells recovered from the decidua or paraaortic lymph node three days after Lm ΔactA (10⁷ CFU) infection initiated midgestation (E11.5) for C57BL/6 female mice during allogeneic pregnancy sired by Balb/c-OVA males. Each symbol reflects the data from a single mouse, and these data are representative of at least three independent experiments each with similar results. Bar, mean±one standard error.

FIG. 8 shows that a CXCR3 blockade initiated before or shortly after virulent Lm prenatal infection protects against fetal wastage and mitigates decidual fetal specific CD8⁺ T cell accumulation. (A) Percent fetal resorption, number of live pups, and frequency of Lm recovery from each concepti five days after virulent Lm (10⁴ CFU) infection initiated midgestation (E11.5) among C57BL/6 female mice bearing allogeneic pregnancy sired by Balb/c males administered anti-CXCR3 antibody (500 μg/mouse) 24 hours before, or 12 or 24 hours after infection compared with isotype or no antibody treated controls. (B) Representative FACS plots and composite data showing percent fetal-OVA₂₅₇₋₂₆₄-specific (CD90.1⁺) among CD8⁺ T cells recovered from the decidua three days after virulent Lm (10⁴ CFU) infection initiated midgestation (E11.5) among C57BL/6 female mice bearing allogeneic pregnancy sired by Balb/c-OVA males administered anti-CXCR3 antibody (500 μg/mouse) 24 hours before, or 12 or 24 hours after infection compared with isotype or no antibody treated controls. (C) Mean fluorescent intensity (MFI) after staining with anti-CXCL9 compared with isotype control antibody among neutrophils (CD11b⁺ Ly6C^{int}) (top) and macrophage (F4/80⁺ CD11b⁻) (bottom) cells recovered from the decidua 3 days after virulent Lm (10⁴ CFU) infection for mice described in panel B. Each symbol reflects the data from a single mouse, and these data are representative of at least three independent experiments each with similar results. Bar, mean±one standard error.

FIG. 9 is a series of graphs showing that ampicillin administration early after virulent Lm prenatal infection protects against fetal wastage. Percent fetal resorption (top), number of live pups (middle), and frequency of Lm recovery from each concepti (bottom) five days after virulent Lm (10⁴ CFU) infection initiated midgestation (E11.5) among female C57BL/6 female mice bearing allogeneic pregnancy sired by Balb/c males administered ampicillin in the drinking water beginning 12 or 24 hours after infection compared with controls maintained on autoclaved drinking water without supplementation. Each symbol reflects the data from a single mouse, and these data are representative of at least three independent experiments each with similar results. Bar, mean±one standard error.

FIG. 10 shows that a CXCR3 blockade protects against fetal wastage and decidual fetal-specific CD8⁺ T cell accumulation triggered by partial depletion of maternal Foxp3⁺ Tregs. (A) Mean fluorescent intensity (MFI) after staining with anti-CXCL9 compared with isotype control antibody among neutrophils (CD11b⁺ Ly6C^{int}) (top) and macrophage (F4/80⁺ CD11b⁻) (bottom) cells recovered from the decidua three days after initiating DT treatment to E11.5 Foxp3^{DTR/WT} female mice on the C57BL/6 background bearing allogeneic pregnancy sired by Balb/-OVA males. (B) Representative FACS plots and composite data showing percent fetal-OVA₂₅₇₋₂₆₄-specific (CD90.1⁺) among CD8⁺ T cells recovered from the decidua three days after initiating DT treatment compared with no DT controls for mice described in panel A. (C) Percent fetal resorption and number of live pups for Foxp3^{DTR/WT} female mice on the C57BL/6 background bearing allogeneic pregnancy sired by Balb/c males administered anti-CXCR3 antibody (500 μg/mouse) 24 hours before, or 12 or 24 hours after initiating sustained daily DT treatment E11.5 compared with no DT or no anti-CXCR3 antibody treatment controls. Each symbol reflects the data from a single mouse, and these data are representative of at least three independent experiments each with similar results. Bar, mean±one standard error.

FIG. 11 shows that pregnancy does not impact the efficiency of antibody mediated T cell depletion. Percent CD4⁺ and CD8⁺ cells among splenocytes from C57BL/6 female mice before mating (virgin) or during allogeneic pregnancy (E11.5) sired by Balb/c males one day after administration of anti-CD4 (clone GK1.5) and anti-CD8α (clone 2.43) antibodies (500 μg/mouse), followed by staining with non-overlapping anti-CD4 (RM4-4) and anti-CD8β (ebioH35-17.2) antibody clones.

FIG. 12 depicts intravascular staining for maternal CD8⁺ T cells with fetal-OVA specificity. Representative histogram plots showing staining by intravenously injected anti-CD45.2 antibody for CD8⁺ T cells recovered from each tissue (top). Representative FACS plots and composite data showing percent fetal-OVA₂₅₇₋₂₆₄-specific (CD90.1⁺) for each source of CD8⁺ T cells three days after Lm ΔactA (10⁷ CFU) infection initiated midgestation (E11.5) among C57BL/6 females during allogeneic pregnancy sired by Balb/c-OVA males (bottom). Each symbol reflects the data from a single mouse, and these data are representative of at least three independent experiments each with similar results. Bar, mean±1 SEM.

FIG. 13 shows the gating strategy for identification of leukocyte subsets among decidual cells. Representative FACS plots illustrate analysis of bulk decidual cells (live cell gate) from C57BL/6 female mice E14.5 during allogeneic pregnancy sired by Balb/c males, elimination of doublets (singlet cell gate), and leukocyte gate (based on expression of the pan-leukocyte marker CD45). Thereafter the composition of each cell subset is identified as follows: neutrophils (CD11b⁺ Ly6C^{int}), macrophages (F4/80⁺ CD11b⁻); natural killer cells (NK1.1⁺ CD4⁻ CD8⁻); B cells (B220⁺ CD4⁻ CD8⁻); CD8⁺ cells (CD8⁺ CD4⁻) and CD4⁺ cells (CD4⁺ CD8⁻).

FIG. 14 is a series of graphs showing discordant tissue tropism between Listeria monocytogenes and influenza A after prenatal infection. (A) Recoverable Lm colony forming units (CFUs) from each tissue five days after infection (10⁴ Lm strain 10403s) initiated midgestation (E11.5) among C57BL/6 female mice during allogeneic pregnancy sired by Balb/c males. (B) Recoverable influenza A virus plaque forming units (PFUs) from each tissue five days after infection (10³ influenza A H1N1 strain PR8) initiated midgestation (E11.5) among C57BL/6 female mice during allogeneic pregnancy sired by Balb/c males. (C) Percent fetal resorption and number of live pups five days after of influenza A infection (10³ influenza A H1N1 strain PR8) for the mice described in panel B. This influenza A inoculum used for infection represents the highest dose that does not cause lethal infection (LD₁₀₀=6000 PFU for non-pregnant control mice). Each symbol reflects the data from a single mouse, and are representative of at least two independent experiments each with similar results. Bar, mean±1 SEM. LOD, limit of detection.

FIG. 15 shows that the reduced susceptibility to Lm infection induced fetal wastage during syngeneic pregnancy is not mitigated by CXCR3 blockade. Percent fetal resorption (top) and concepti with recoverable bacteria (bottom) five days after infection with virulent Lm (strain 10403s) at the indicated dosages initiated midgestation (E11.5) among C57BL/6 females bearing either allogeneic pregnancy sired by Balb/c males or syngeneic pregnancy sired by isogenic C57BL/6 males, and administered either anti-CXCR3 antibody or isotype control antibody (500 μg/mouse) 24 hours before infection. Each symbol reflects the data from a single mouse, and these data are representative of at least three independent experiments each with similar results. Bar, mean±1 SEM.

### DETAILED DESCRIPTION OF THE INVENTION

Mammalian pregnancy requires protection against immunological rejection of the developing fetus bearing discordant paternal antigens. Immune evasion in this developmental context entails silenced expression of chemoattractant ‘chemokine’ proteins that prevents harmful immune cells from penetrating the maternal-fetal interface. As described herein, it was found that fetal wastage triggered by prenatal Listeria monocytogenes (Lm) infection was driven by placental recruitment of CXCL9 chemokine producing inflammatory neutrophil and macrophage cells that unleashed fetal-specific T cell infiltration into the decidua. The study herein showed that Maternal CD8⁺ T cells with fetal specificity upregulated expression of the chemokine receptor, CXCR3, and together with neutrophil and macrophage cells, were essential for infection induced fetal resorption. Reciprocally, decidual accumulation of maternal T cells with fetal specificity and infection induced fetal wastage were extinguished by CXCR3 blockade or in CXCR3-deficient mice. The study herein further shows that protection against fetal wastage and in utero Lm invasion was maintained even when CXCR3 neutralization was initiated after infection, and extended to fetal resorption triggered by partial ablation of immune suppressive maternal regulatory T cells that expand during pregnancy to sustain fetal tolerance. As a result, neutralizing the CXCR3 pathway is useful for mitigating immune-mediated pregnancy complications, such as fetal wastage, which refers to the loss of a fetus through spontaneous abortion or stillbirth. In humans, fetal wastage may occur between the 20^{th} week of pregnancy and the 28^{th} day of life of a fetus.

Accordingly, the present disclosure provides compositions and methods for reducing the risk of or preventing fetal wastage using a CXCR3 inhibitor.

### CXCR3 Inhibitors

CXCR3 (Chemokine (C-X-C motif) receptor 3) is a receptor in the CXC chemokine receptor family. There are at least two variants of CXCR3:(1) CXCR3-A (also called isoform 1), which can bind to CXCL9 (MIG), CXCL10 (IP-10), and CXCL11 (I-TAC), and (2) CXCR3-B (also called isoform 2), which can bind to CXCL4, CXCL9, CXCL10, and CXCL11.

Exemplary human CXCR3 protein and mRNA sequences are provided below. The mRNA sequences are derived from cDNA, and thus it is to be understood that the “T”s in the mRNA sequences below may be replaced with “U”s.

>gi|4504098|ref|NM_001504.1| Homo sapiens
chemokine (C-X-C motif) receptor 3 (CXCR3),
transcript variant 1, mRNA
(SEQ ID NO: 1)
CCAACCACAAGCACCAAAGCAGAGGGGCAGGCAGCACACCACCCAGCAG
CCAGAGCACCAGCCCAGCCATGGTCCTTGAGGTGAGTGACCACCAAGTG
CTAAATGACGCCGAGGTTGCCGCCCTCCTGGAGAACTTCAGCTCTTCCT
ATGACTATGGAGAAAACGAGAGTGACTCGTGCTGTACCTCCCCGCCCTG
CCCACAGGACTTCAGCCTGAACTTCGACCGGGCCTTCCTGCCAGCCCTC
TACAGCCTCCTCTTTCTGCTGGGGCTGCTGGGCAACGGCGCGGTGGCAG
CCGTGCTGCTGAGCCGGCGGACAGCCCTGAGCAGCACCGACACCTTCCT
GCTCCACCTAGCTGTAGCAGACACGCTGCTGGTGCTGACACTGCCGCTC
TGGGCAGTGGACGCTGCCGTCCAGTGGGTCTTTGGCTCTGGCCTCTGCA
AAGTGGCAGGTGCCCTCTTCAACATCAACTTCTACGCAGGAGCCCTCCT
GCTGGCCTGCATCAGCTTTGACCGCTACCTGAACATAGTTCATGCCACC
CAGCTCTACCGCCGGGGGCCCCCGGCCCGCGTGACCCTCACCTGCCTGG
CTGTCTGGGGGCTCTGCCTGCTTTTCGCCCTCCCAGACTTCATCTTCCT
GTCGGCCCACCACGACGAGCGCCTCAACGCCACCCACTGCCAATACAAC
TTCCCACAGGTGGGCCGCACGGCTCTGCGGGTGCTGCAGCTGGTGGCTG
GCTTTCTGCTGCCCCTGCTGGTCATGGCCTACTGCTATGCCCACATCCT
GGCCGTGCTGCTGGTTTCCAGGGGCCAGCGGCGCCTGCGGGCCATGCGG
CTGGTGGTGGTGGTCGTGGTGGCCTTTGCCCTCTGCTGGACCCCCTATC
ACCTGGTGGTGCTGGTGGACATCCTCATGGACCTGGGCGCTTTGGCCCG
CAACTGTGGCCGAGAAAGCAGGGTAGACGTGGCCAAGTCGGTCACCTCA
GGCCTGGGCTACATGCACTGCTGCCTCAACCCGCTGCTCTATGCCTTTG
TAGGGGTCAAGTTCCGGGAGCGGATGTGGATGCTGCTCTTGCGCCTGGG
CTGCCCCAACCAGAGAGGGCTCCAGAGGCAGCCATCGTCTTCCCGCCGG
GATTCATCCTGGTCTGAGACCTCAGAGGCCTCCTACTCGGGCTTGTGAG
GCCGGAATCCGGGCTCCCCTTTCGCCCACAGTCTGACTTCCCCGCATTC
CAGGCTCCTCCCTCCCTCTGCCGGCTCTGGCTCTCCCCAATATCCTCGC
TCCCGGGACTCACTGGCAGCCCCAGCACCACCAGGTCTCCCGGGAAGCC
ACCCTCCCAGCTCTGAGGACTGCACCATTGCTGCTCCTTAGCTGCCAAG
CCCCATCCTGCCGCCCGAGGTGGCTGCTGGAGCCCCACTGCCCTTCTCA
TTTGGAAACTAAAACTTCATCTTCCCCAAGTGCGGGGAGTACAAGGCAT
GGCGTAGAGGGTGCTGCCCCATGAAGCCACAGCCCAGGCCTCCAGCTCA
GCAGTGACTGTGGCCATGGCCCCAAGACCTCTATATTTGCTCTTTTATT
TTTATGTCTAAAATCCTGCTTAAAACTTTTCAATAAACAAGATCGTCAG
GACCAAAAAAAAAAAAAAAAAAAAAAAAAAAAAAAAAAAAAAAAAAAAA
AA
>gi|4504099|ref|NP_001495.1| C-X-C chemokine
receptor type 3 isoform 1 [Homo sapiens]
(SEQ ID NO: 2)
MVLEVSDHQVLNDAEVAALLENFSSSYDYGENESDSCCTSPPCPQDFSL
NFDRAFLPALYSLLFLLGLLGNGAVAAVLLSRRTALSSTDTFLLHLAVA
DTLLVLTLPLWAVDAAVQWVFGSGLCKVAGALFNINFYAGALLLACISF
DRYLNIVHATQLYRRGPPARVTLTCLAVWGLCLLFALPDFIFLSAHHDE
RLNATHCQYNFPQVGRTALRVLQLVAGFLLPLLVMAYCYAHILAVLLVS
RGQRRLRAMRLVVVVVVAFALCWTPYHLVVLVDILMDLGALARNCGRES
RVDVAKSVTSGLGYMHCCLNPLLYAFVGVKFRERMWMLLLRLGCPNQRG
LQRQPSSSRRDSSWSETSEASYSGL
>gi|218563729|ref|NM_001142797.1| Homo sapiens
chemokine (C-X-C motif) receptor 3 (CXCR3),
transcript variant 2, mRNA
(SEQ ID NO: 3)
CCAACCACAAGCACCAAAGCAGAGGGGCAGGCAGCACACCACCCAGCAG
CCAGAGCACCAGCCCAGCCATGGTCCTTGAGGGGTCCCTGGGCCGATGG
GATCACGCAGAAGAATGCGAGAGAAGCAGCCTTTGAGAAGGGAAGTCAC
TATCCCAGAGCCCAGGCTGAGCGGATGGAGTTGAGGAAGTACGGCCCTG
GAAGACTGGCGGGGACAGTTATAGGAGGAGCTGCTCAGAGTAAATCACA
GACTAAATCAGACTCAATCACAAAAGAGTTCCTGCCAGGCCTTTACACA
GCCCCTTCCTCCCCGTTCCCGCCCTCACAGGTGAGTGACCACCAAGTGC
TAAATGACGCCGAGGTTGCCGCCCTCCTGGAGAACTTCAGCTCTTCCTA
TGACTATGGAGAAAACGAGAGTGACTCGTGCTGTACCTCCCCGCCCTGC
CCACAGGACTTCAGCCTGAACTTCGACCGGGCCTTCCTGCCAGCCCTCT
ACAGCCTCCTCTTTCTGCTGGGGCTGCTGGGCAACGGCGCGGTGGCAGC
CGTGCTGCTGAGCCGGCGGACAGCCCTGAGCAGCACCGACACCTTCCTG
CTCCACCTAGCTGTAGCAGACACGCTGCTGGTGCTGACACTGCCGCTCT
GGGCAGTGGACGCTGCCGTCCAGTGGGTCTTTGGCTCTGGCCTCTGCAA
AGTGGCAGGTGCCCTCTTCAACATCAACTTCTACGCAGGAGCCCTCCTG
CTGGCCTGCATCAGCTTTGACCGCTACCTGAACATAGTTCATGCCACCC
AGCTCTACCGCCGGGGGCCCCCGGCCCGCGTGACCCTCACCTGCCTGGC
TGTCTGGGGGCTCTGCCTGCTTTTCGCCCTCCCAGACTTCATCTTCCTG
TCGGCCCACCACGACGAGCGCCTCAACGCCACCCACTGCCAATACAACT
TCCCACAGGTGGGCCGCACGGCTCTGCGGGTGCTGCAGCTGGTGGCTGG
CTTTCTGCTGCCCCTGCTGGTCATGGCCTACTGCTATGCCCACATCCTG
GCCGTGCTGCTGGTTTCCAGGGGCCAGCGGCGCCTGCGGGCCATGCGGC
TGGTGGTGGTGGTCGTGGTGGCCTTTGCCCTCTGCTGGACCCCCTATCA
CCTGGTGGTGCTGGTGGACATCCTCATGGACCTGGGCGCTTTGGCCCGC
AACTGTGGCCGAGAAAGCAGGGTAGACGTGGCCAAGTCGGTCACCTCAG
GCCTGGGCTACATGCACTGCTGCCTCAACCCGCTGCTCTATGCCTTTGT
AGGGGTCAAGTTCCGGGAGCGGATGTGGATGCTGCTCTTGCGCCTGGGC
TGCCCCAACCAGAGAGGGCTCCAGAGGCAGCCATCGTCTTCCCGCCGGG
ATTCATCCTGGTCTGAGACCTCAGAGGCCTCCTACTCGGGCTTGTGAGG
CCGGAATCCGGGCTCCCCTTTCGCCCACAGTCTGACTTCCCCGCATTCC
AGGCTCCTCCCTCCCTCTGCCGGCTCTGGCTCTCCCCAATATCCTCGCT
CCCGGGACTCACTGGCAGCCCCAGCACCACCAGGTCTCCCGGGAAGCCA
CCCTCCCAGCTCTGAGGACTGCACCATTGCTGCTCCTTAGCTGCCAAGC
CCCATCCTGCCGCCCGAGGTGGCTGCCTGGAGCCCCACTGCCCTTCTCA
TTTGGAAACTAAAACTTCATCTTCCCCAAGTGCGGGGAGTACAAGGCAT
GGCGTAGAGGGTGCTGCCCCATGAAGCCACAGCCCAGGCCTCCAGCTCA
GCAGTGACTGTGGCCATGGTCCCCAAGACCTCTATATTTGCTCTTTTAT
TTTTATGTCTAAAATCCTGCTTAAAACTTTTCAATAAACAAGATCGTCA
GGACCAAAAAAAAAAAAAAAAAAAAAAAAAAAAAAAAAAAAAAAAAAAA
AAA
>gi|218563730|ref|NP_001136269.1| C-X-C chemokine
receptor type 3 isoform 2 [homo sapiens]
(SEQ ID NO: 4)
MELRKYGPGRLAGTVIGGAAQSKSQTKSDSITKEFLPGLYTAPSSPFPP
SQVSDHQVLNDAEVAALLENFSSSYDYGENESDSCCTSPPCPQDFSLNF
DRAFLPALYSLLFLLGLLGNGAVAAVLLSRRTALSSTDTFLLHLAVADT
LLVLTLPLWAVDAAVQWVFGSGLCKVAGALFNINFYAGALLLACISFDR
YLNIVHATQLYRRGPPARVTLTCLAVWGLCLLFALPDFIFLSAHHDERL
NATHCQYNFPQVGRTALRVLQLVAGFLLPLLVMAYCYAHILAVLLVSRG
QRRLRAMRLVVVVVVAFALCWTPYHLVVLVDILMDLGALARNCGRESRV
DVAKSVTSGLGYMHCCLNPLLYAFVGVKFRERMWMLLLRLGCPNQRGLQ
RQPSSSRRDSSWSETSEASYSGL

A CXCR3 inhibitor is an agent capable of reducing or disrupting the CXCR3 signaling in a cell. The CXCR3 inhibitor disclosed herein may be an agent (e.g., an antibody or a small molecule) that interferes the interaction between CXCR3 and a ligand thereof, such as CXCL4, CXCL9, CXCL10, and/or CXCL11. The CXCR3 inhibitor may also be an agent (e.g., a inhibitory polynucleotide or oligonucleotide such as interfering RNA or antisense oligonucleotide) that suppresses CXCR3 transcription and/or translation, thereby reducing the mRNA/protein level of this receptor. The CXCR3 inhibitor as described herein may reduce the CXCR3 signaling in cells by at least 20%, e.g., 30%, 40%, 50%, 60%, 70%, 80%, 90%, 95% or above. The inhibitory activity of such an inhibitor against CXCR3 can be determined by conventional methods, e.g., the CXCR3 bioassay method disclosed in US 20100305088.

In some embodiments, the CXCR3 inhibitor is an antibody that specifically binds to CXCR3 and neutralizes its activity. As used herein, the term “antibody” as includes but is not limited to polyclonal, monoclonal, humanized, chimeric, Fab fragments, Fv fragments, F(ab′) fragments and F(ab′)₂ fragments, as well as single chain antibodies (scFv), fusion proteins and other synthetic proteins which comprise the antigen-binding site of the antibody.

Antibodies can be made by the skilled person using methods and commercially available services and kits known in the art. Methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013)).

Antibodies may be produced by standard techniques, for example by immunization with the appropriate polypeptide or portion(s) thereof, or by using a phage display library. If polyclonal antibodies are desired, a selected mammal (e.g., mouse, rabbit, goat, horse, etc) is immunized with an immunogenic polypeptide bearing a desired epitope(s), optionally haptenized to another polypeptide. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Serum from the immunized animal is collected and treated according to known procedures. If serum containing polyclonal antibodies to the desired epitope contains antibodies to other antigens, the polyclonal antibodies can be purified by immunoaffinity chromatography or any other method known in the art. Techniques for producing and processing polyclonal antisera are well known in the art.

An antibody specifically binds to CXCR3 if the antibody binds CXCR3 with a greater affinity than for an irrelevant polypeptide. Preferably, the antibody binds CXCR3 with at least 5, or at least 10 or at least 50 times greater affinity than for the irrelevant polypeptide. More preferably, the antibody molecule binds CXCR3 with at least 100, or at least 1,000, or at least 10,000 times greater affinity than for the irrelevant polypeptide. Such binding may be determined by methods well known in the art, such surface plasmon resonance such as a Biacore® system. In some embodiments, the antibody has an affinity (as measured by a dissociation constant, K_{D}) for CXCR3 of at least 10⁻⁷ M, 10⁻⁸ M, 10⁻⁹ M, 10⁻¹⁰ M, or 10⁻¹¹ M.

Anti-CXCR3 antibodies are commercially available. See, e.g., products from Abcam: Anti-CXCR3 antibody [EPR7469(B)] (ab154845), Anti-CXCR3 antibody [2Ar1] (ab64714), Anti-CXCR3 antibody [106] (ab125255), Anti-CXCR3 antibody (ab71864), Anti-CXCR3 antibody (ab133420), Anti-CXCR3 antibody (ab77907), Anti-CXCR3 antibody—N-terminal (ab154033), Anti-CXCR3 antibody [MM0223-7K22] (ab89255), and Anti-CXCR3 antibody [49801.111] (ab10402); products from R&D systems: Human CXCR3 MAb (Clone 49801, MAB160-100); products from StemCell: Anti-Human CD183 (CXCR3) Antibody, Clone G025H7 (60088); products from Thermo Scientific: CXCR3/CD183 Antibody (PA5-19828); and products from BioXcell: InVivoMAb anti m CD183 (CXCR3-173). Other anti-CXCR3 antibodies are disclosed, e.g., in PCT Publication No. WO2013109974, WO2008094942, WO 2001072334, WO2005030793 each of which are incorporated by reference herein in their entirety.

In some examples, the anti-CXCR3 antibodies described herein are full human antibodies, which can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455, and. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

In other examples, the anti-CXCR3 antibodies are humanized antibodies. Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Nall. Acad. Sci. USA, 86:10029-10033 (1989).

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the CXCR3 inhibitor is an interfering RNA such as a small interfering RNA (siRNA) short hairpin RNA (shRNA). In some embodiments, the CXCR3 inhibitor is a small interfering RNA (siRNA) that binds to a CXCR3 mRNA and blocks its translation or degrades the mRNA via RNA interference. Exemplary small interfering RNAs are described by Hannon et al. Nature, 418 (6894): 244-51 (2002); Brummelkamp et al., Science 21, 21 (2002); and Sui et al., Proc. Natl Acad. Sci. USA 99, 5515-5520 (2002). RNA interference (RNAi) is the process of sequence-specific post-transcriptional gene silencing in animals initiated by double-stranded (dsRNA) that is homologous in sequence to the silenced gene. siRNAs are generally RNA duplexes with each strand being 20-25 (such as 19-21) base pairs in length. In some embodiments, the CXCR3 inhibitor is a short hairpin RNA (shRNA) that is complementary to a CXCR3 nucleic acid (e.g., a CXCR3 mRNA). An shRNA typically contains of a stem of 19-29 base pairs, a loop of at least 4 nucleotides (nt), and optionally a dinucleotide overhang at the 3′ end. Expression of shRNA in a subject can be obtained by delivery of a vector (e.g., a plasmid or viral or bacterial vectors) encoding the shRNA. siRNAs and shRNAs may be designed using any method known in the art or commercially available (see, e.g., products available from Dharmacon and Life Technologies). An siRNA may also comprise one or more chemical modifications, such as a base modification and/or a bond modification to at least improve its stability and binding affinity to the target mRNA.

In some embodiments, the CXCR3 inhibitor is an antisense oligonucleotide that is complementary to a CXCR3 nucleic acid (e.g., a CXCR3 mRNA). Antisense oligonucleotides are generally single-stranded nucleic acids (either a DNA, RNA, or hybrid RNA-DNA molecule), which are complementary to a target nucleic acid sequence, such as a portion of a CXCR3 mRNA. By binding to the target sequence, an RNA-RNA, a DNA-DNA, or RNA-DNA duplex is formed, thereby inhibiting the function or level of the target nucleic acid, such as by blocking the transcription, processing, poly(A) addition, replication, translation, or promoting inhibitory mechanisms of the cells, such as promoting mRNA degradation. In some embodiments, an antisense oligonucleotide is 10 to 40, 12 to 35, or 15 to 35 bases in length, or any integer in between. An antisense oligonucleotide can comprise one or more modified bases, such as 2-Aminopurine, 2,6-Diaminopurine (2-Amino-dA), 5-Bromo dU, 5-Methyl dC, deoxylnosine, Locked Nucleic Acid (LNA), 5-Nitroindole, 2′-O-Methyl bases, Hydroxmethyl dC, 2′ Fluoro bases. An antisense oligonucleotide can comprise one or more modified bonds, such as a phosphorothioate bond.

In some embodiments, the CXCR3 inhibitor is a ribozyme that is complementary to a CXCR3 nucleic acid (e.g., a CXCR3 mRNA) and cleaves the CXCR3 nucleic acid. Ribozymes are RNA or RNA-protein complexes that cleave nucleic acids in a site-specific fashion. Ribozymes have specific catalytic domains that possess endonuclease activity. The ribozymes of the present disclosure may be synthetic ribozymes, such as those described in U.S. Pat. No. 5,254,678. These synthetic ribozymes have separate hybridizing regions and catalytic regions; therefore, the hybridizing regions can be designed to recognize a target sequence, such as a CXCR3 sequence as described herein.

siRNAs, shRNAs, ribozymes, and antisense oligonucleotides as described herein may be complementary to a CXCR3 nucleic acid (e.g., a CXCR3 mRNA), or a portion thereof. It is to be understood that complementarity includes 100% complementarity but does not necessarily exclude mismatches at one or more locations, resulting in, e.g., at least 80%, at least 90%, at least 95%, at least 98%, or at least 99% complementarity.

In some embodiments, the CXCR3 inhibitor is a non-antibody peptide or protein. The peptide or protein may comprise an amino acid sequence that interferes with the activity of CXCR3, such as by competing with a natural ligand for CXCR3, e.g., competing with CXCL9. Proteins and peptides may be designed using any method known in the art, e.g., by screening libraries of proteins or peptides for binding to CXCR3 or inhibition of CXCR3 binding to a ligand, such as CXCL9.

When applicable, the CXCR3 inhibitor can be expressed from a vector, which may be used for delivering the CXCR3 into a subject who needs the treatment. A “vector”, as used herein is any vehicle capable of facilitating the transfer of a CXCR3 inhibitor (e.g., a shRNA, siRNA, ribozyme, antisense oligonucleotide, protein, peptide, or antibody) to a cell in the subject, such as a cell expressing CXCR3 receptor. In general, vectors include, but are not limited to, plasmids, phagemids, viruses, and other vehicles derived from viral or bacterial sources that have been manipulated by the insertion or incorporation of a sequence encoding a CXCR3 inhibitor. Viral vectors include, but are not limited to nucleic acid sequences from the following viruses: retrovirus; lentivirus; adenovirus; adeno-associated virus; SV40-type viruses; polyoma viruses; Epstein-Barr viruses; papilloma viruses; herpes virus; vaccinia virus; polio virus. One can readily employ other vectors not named but known to the art.

Viral vectors may be based on non-cytopathic eukaryotic viruses in which nonessential genes have been replaced with a sequence encoding a CXCR3 inhibitor. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are known in the art.

Other viral vectors include adeno-viruses and adeno-associated viruses, which are double-stranded DNA viruses that have also been approved for human use in gene therapy. The adeno-associated virus can be engineered to be replication deficient and is capable of infecting a wide range of cell types and species.

Other vectors include plasmid vectors. Plasmid vectors have been extensively described in the art and are well known to those of skill in the art. See, e.g., Sambrook et al. Molecular Cloning: A Laboratory Manual. Cold Spring Harbor Laboratory Press; 4th edition (Jun. 15, 2012). Exemplary plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA, such as a sequence encoding a CXCR3 inhibitor.

In some embodiments, CXCR3 inhibitor nucleic acid sequence is under the control of a heterologous regulatory region, e.g., a heterologous promoter. The promoter can be, e.g., a ubiquitous promoter such as a CMV promoter, ActB promoter, or Ubiquitin B promoter. The promoter can also be a tissue-specific promoter or synthetic promoter. Promoters are well known in the art and commercially available (see, e.g., products available from InvivoGen).

In some embodiments, the CXCR3 inhibitor is a small molecule, such as a small organic molecule, which typically has a molecular weight less than 5,000 kDa. Suitable small molecules include those that bind to CXCR3, or a fragment thereof, and may be identified by methods such as screening large libraries of compounds (Beck-Sickinger & Weber (2001) Combinational Strategies in Biology and Chemistry (John Wiley & Sons, Chichester, Sussex); by structure-activity relationship by nuclear magnetic resonance (Shuker et al (1996) “Discovering high-affinity ligands for proteins: SAR by NMR. Science 274: 1531-1534); encoded self-assembling chemical libraries Melkko et al (2004) “Encoded self-assembling chemical libraries.” Nature Biotechnol. 22: 568-574); DNA-templated chemistry (Gartner et al (2004) “DNA-tem plated organic synthesis and selection of a library of macrocycles. Science 305: 1601-1605); dynamic combinatorial chemistry (Ramstrom & Lehn (2002) “Drug discovery by dynamic combinatorial libraries.” Nature Rev. Drug Discov. 1: 26-36); tethering (Arkin & Wells (2004) “Small-molecule inhibitors of protein-protein interactions: progressing towards the dream. Nature Rev. Drug Discov. 3: 301-317); and speed screen (Muckenschnabel ei al (2004) “SpeedScreen: label-free liquid chromatography-mass spectrometry-based high-throughput screening for the discovery of orphan protein ligands.” Anal. Biochem. 324: 241-249). Typically, small molecules will have a dissociation constant for CXCR3 in the nanomolar range.

The capability of a candidate compound, such as a small molecule, protein, or peptide, to bind to or interact with a CXCR3 polypeptide or fragment thereof may be measured by any method of detecting/measuring a protein/protein interaction or other compound/protein interaction. Suitable methods include methods such as, for example, yeast two-hybrid interactions, co-purification, ELISA, co-immunoprecipitation and surface plasmon resonance methods. Thus, the candidate compound may be considered capable of binding to the polypeptide or fragment thereof if an interaction may be detected between the candidate compound and the polypeptide or fragment thereof by ELISA, co-immunoprecipitation or surface plasmon resonance methods or by a yeast two-hybrid interaction or copurification method, all of which are known in the art. Screening assays which are capable of high throughput operation are also contemplated. Examples may include cell based assays and protein-protein binding assays.

Exemplary CXCR3 inhibitor small molecules include a 3-(amido or sulphamido)-4-(4-substituted-azinyl)benzamide or benzsulphonamide compounds (see, e.g., WO 2009105435), a dihydro-quinazoline analog (see, e.g., Pease J. E. el al, 2009, Expert Opin. Ther. or Liu J et al, 2009) like AMG487 (see, e.g., Jiwen Liu, et al, 2009, An-Rong Lia et al, 2008 or Johnson M. et al, 2007), a piperidinyl-urea derivative (see, e.g., Pease J. E. el al, 2009, Expert Opin. Ther.) like a 1-aryl-3-piperidin-4-yl-urea derivative (see, e.g., Allen et AL, 2007) or a 5-(piperidin-4-yl)amino-1,2,4-thiadiazole derivative (see, e.g., Watson et AL, 2007), or a tropenyl derivative (see, e.g., Watson et AL, 2008) or a 2-aminoquinoline substituted piperidines derivative (see, e.g., Knight et AL, 2008), a 4-aryl-[1,4]diazepine ethyl ureas derivative (see, e.g., Pease J. E. el al, 2009, Expert Opin. Ther. and Cole A G, et al., 2006), a benzimidazole derivative or a 2-iminobenzimidazole (see, e.g., Pease J. E. el al, 2009, Expert Opin. Ther., Hayes M E, Wallace G A, et AL, 2008 and Hayes M E, Breinlinger E C et AL, 2008), a benzetimide derivative (see, e.g., Pease J. E. el al, 2009, Expert Opin. Ther. and Bongartz J P et al., 2008), a benzetimide derivative (see, e.g., Pease J. E. el al, 2009, Expert Opin. Ther. and Bongartz J P et al., 2008), an ergo line derivative (see, e.g., Thoma G. et AL, 2009 or Choudhary M S et AL, 1995 and patent application WO2006128658), a heterocyclic substituted piperazine derivative (see, e.g., patent applications WO2006088837 and WO2008008453), a heterocyclic substituted pyridine derivative (see, e.g., patent applications WO2007109238 and WO2006088840), a imidazolium derivative (see, e.g., Verzijl D. Et al., and patent application WO03101970), an imidazole derivative (see, e.g., Du X. et Al, 2008), a piperazinyl-piperidine derivative (see, e.g., McGuinness B F et AL, 2009), a Camphor sulfonamide derivative (see, e.g., Wang Y et AL, 2009), a pyrazinyl substituted piperazine-piperidine derivative (see, e.g., patent application WO2006088921), a pyridyl and phenyl substituted piperazine-piperidine derivative (see, e.g., patent application WO2006088919), a piperazine-piperidine derivative (see, e.g., patent application WO2006088836), a heteroaryl substituted pyrazinyl-piperazine-piperidine derivative (see, e.g., patent application WO2006091428), an amine-linked pyridyl and phenyl substituted piperazine-piperidine derivative (see, e.g., patent application WO2006088920, a thiazole derivative (see, e.g., patent application WO2007064553), a substituted heterocyclic derivative (see, e.g., patent application WO2007047202), NBI74330 (see, e.g., Jopling L A et AL, 2007), AMG487 (see, e.g., Jiwen Liu, et al, 2009, An-Rong Lia et al, 2008 or Johnson M. et al, 2007), or AMG1237845 (see, e.g., Rosenblum J M et al, 2009). Each of the above-reference patent publications or references is incorporated herein by reference.

Other exemplary small molecule inhibitors of CXCR3 include 3H-pyrido[2,3-d]pyrimidin-4-one derivatives N-1R-[3-(4-ethoxy-phenyl)-4-oxo-3,4-dihydro-pyrido[2,3-d]pyrimidin-2-yl]-ethyl-N-pyridin-3-ylmethyl-2-(4-fluoro-3-trifluoromethyl-phenyl)-acetamide (VUF10472/NBI-74330) and N-1R-[3-(4-ethoxy-phenyl)-4-oxo-3,4-dihydro-pyrido[2,3-d]pyrimidin-2-yl]-ethyl-N-pyridin-3-ylmethyl-2-(4-trifluoromethoxy-phenyl)-acetamide (VUF10085/AMG-487), the 3H-quinazolin-4-one decanoic acid {1-[3-(4-cyano-phenyl)-4-oxo-3,4-dihydro-quinazolin-2-yl]-ethyl}-(2-dimethylamino-ethyl)-amide (VUF5834), the imidazolium compound 1,3-bis-[2-(3,4-dichloro-phenyl)-2-oxo-ethyl]-3H-imidazol-1-ium bromide (VUF10132), and the quaternary ammonium anilide N,N-dimethyl-N-[4-[[[2-(4-methylphenyl)-6,7-dihydro-5H-benzocyclohepten-8-yl]-carbonyl]amino]benzyl] tetrahydro-2H-pyran-4-aminium chloride (TAK-779) (see, e.g., Verzijl et al. Noncompetitive Antagonism and Inverse Agonism as Mechanism of Action of Nonpeptidergic Antagonists at Primate and Rodent CXCR3 Chemokine Receptors. J Pharmacol Exp Ther. 2008 May; 325(2):544-55).

Other small molecules, antibodies and other agents that bind to CXCR3 receptor and modulate its activity are described in U.S. Pat. No. 7,622,264, U.S. Pat. No. 7,541,435, U.S. Pat. No. 7,427,487, U.S. Pat. No. 7,407,655, U.S. Pat. No. 7,405,275, U.S. Pat. No. 7,378,524, U.S. Pat. No. 7,332,294, U.S. Pat. No. 7,244,555, U.S. Pat. No. 7,183,413, U.S. Pat. No. 7,138,229, U.S. Pat. No. 7,029,862, U.S. Pat. No. 6,992,084, U.S. Pat. No. 6,559,160, U.S. Pat. No. 6,184,358, and U.S. Pat. No. 6,140,064, the relevant disclosures of which are incorporated by reference herein for the intended purpose.

### Formulations and Routes of Administration

In some embodiments, the CXCR3 inhibitor is formulated for administration to an subject as a pharmaceutical composition, e.g., together with a pharmaceutically acceptable carrier, diluent or excipient.

A carrier, diluent or excipient that is “pharmaceutically acceptable” includes one that is sterile and pyrogen free. Suitable pharmaceutical carriers, diluents and excipients are well known in the art. The carrier(s) must be “acceptable” in the sense of being compatible with the inhibitor and not deleterious to the recipients thereof.

A pharmaceutical composition comprising any of the CXCR3 inhibitors described herein may be administered by any administration route known in the art, such as parenteral administration, oral administration, buccal administration, sublingual administration, topical administration, or inhalation, in the form of a pharmaceutical formulation comprising the active ingredient, optionally in the form of a non-toxic organic, or inorganic, acid, or base, addition salt, in a pharmaceutically acceptable dosage form. In some embodiments, the administration route is oral administration and the formulation is formulated for oral administration.

In some embodiments, the pharmaceutical compositions or formulations are for parenteral administration, such as intravenous, intra-arterial, intra-muscular, subcutaneous, or intraperitoneal administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well-known to those skilled in the art.

In some embodiments, the pharmaceutical composition or formulation is suitable for oral, buccal or sublingual administration, such as in the form of tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, coloring matter or dyes, with emulsifying and/or suspending agents and with diluents such as water, ethanol, propylene glycol and glycerin, and combinations thereof.

In some embodiments, the pharmaceutical composition or formulation is suitable for intranasal administration or inhalation, such as delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoro-ethane, a hydrofluoroalkane, carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g. using a mixture of ethanol and the propellant as the solvent, which may additionally contain a lubricant. Capsules and cartridges (made, for example, from gelatin) for use in an inhaler or insufflator may be formulated to contain a powder mix of the inhibitor and a suitable powder base such as lactose or starch.

In some embodiments, the pharmaceutical compositions or formulations are suitable for topical administration to a subject. The inhibitor may be applied topically in the form of a lotion, solution, cream, ointment or dusting powder, or may be transdermally administered, for example, by the use of a skin patch. For application topically to the skin, the inhibitor can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water. Formulations suitable for topical administration in the mouth include lozenges comprising the active ingredient; pastilles comprising the active ingredient in an inert basis such as gelatin and glycerin, or sucrose and acacia; and mouth-washes comprising the active ingredient in a suitable liquid carrier.

The formulations may be presented in unit-dose or multi-dose containers, for example sealed ampoules or vials, and may be stored in a freeze-dried (lyophilized) condition requiring only the addition of the sterile liquid carrier immediately prior to use.

In some embodiments, the inhibitor is administered to a subject at a dose of between 0.1 to 1,000 mg per subject or between 0.001 to 10 mg/kg per subject, administered in single or divided doses. A physician in any event may determine the actual dosage which will be most suitable for any subject, which will vary with the age, weight and the particular disease or disorder to be treated or prevented.

Vectors encoding an inhibitor may be administered to a subject using any method known in the art, such as using liposomes, viral vectors (including vaccinia, modified vaccinia, adenovirus, retrovirus, lentivirus, and adeno-associated viral (AAV) vectors), and by direct delivery of the vector.

Also provided herein are compositions for use in reducing the risk of or preventing fetal wastage, the composition comprising a CXCR3 inhibitor as described herein. Also provided herein are uses of a CXCR3 inhibitor as described herein or a composition comprising a CXCR3 inhibitor as described herein in the manufacture of a medicament for reducing the risk of or preventing fetal wastage.

### Therapeutic Applications of CXCR3 Inhibitors

Aspects of the disclosure relate to a method of reducing the risk of fetal wastage or of preventing fetal wastage. In some embodiments, the method comprises administering to a pregnant subject a CXCR3 inhibitor as described herein in an amount effective to reduce the risk of or to prevent fetal wastage.

As used herein, “reducing the risk of fetal wastage” means reducing the risk over the term of pregnancy of the subject of having a spontaneous abortion or stillbirth. As used herein, “prevention of fetal wastage” means preventing the occurrence of fetal wastage during the term of pregnancy of the subject, such that a still birth or abortion is prevented and a live birth occurs.

An “effective amount,” or an “amount effective to”, as used herein, refers to an amount of a CXCR3 inhibitor as described herein that is effective in producing the desired therapeutic, ameliorative, inhibitory or preventative effect, and/or results in a desired clinical effect, such as a live birth and/or inhibition of fetal-specific CD8⁺ T cells in the subject. For example, an effective amount of a CXCR3 inhibitor described herein when administered to a subject results in, e.g., the reduction of the risk of fetal wastage or the prevention of fetal wastage.

Fetal-specific CD8⁺ T cells include maternal CD8⁺ T cells with specificity to fetus-specific antigens, for example, paternal antigens expressed by a fetus. Fetal-specific CD8⁺ cells may be derived from the decidua. Alternatively, they may be derived from blood and/or lymph node.

In some embodiments, the CXCR3 inhibitor as described herein is used in an amount sufficient to inhibits fetal-specific CD8⁺ T cells, which refers to the reduction of the overall fetal-specific CD8⁺ T cells activity, e.g., by at least 20%, 30%, 40%, 50%, 60%, 70%, 80%, 90% or above. Such inhibition can be achieved by, for example, direct inhibition of fetal-specific CD8⁺ T cells, blockade of fetal-specific CD8⁺ cell differentiation and/or maturation, suppression of fetal-specific CD8⁺ T cells activation and proliferation, which would lead to reduced production of inflammatory cytokines (e.g., IFN-γ), and/or blockade of fetal-specific CD8⁺ cell tissue filtration. In some embodiments, the activity of fetal-specific CD8⁺ T cells that are targeted to the placenta, the decidual, and/or the maternal-fetal interface are inhibited, for example, the accumulation and/or infiltration of fetal-specific CD8⁺ T cells into the placenta and/or the decidua, and/or at the maternal-fetal interface, are inhibited.

In some embodiments, the CXCR3 inhibitor is administered at a specific period before, during, or after an infection has occurred in the subject. In some embodiments, the CXCR3 inhibitor is administered prior to manifestation of one or more symptoms of infection (e.g., prior to the presence of one or more symptoms selected from fever (e.g., ranging between 38.2° C. and 41.2° C.), abdominal pain, back pain, vomiting, diarrhea, headache, myalgia, a white blood cell count from 3900 to 33,800 cells/mm³ or sore throat) or prior to an infection (e.g., as detectable by culture of blood, blood, amniotic fluid, spinal fluid or placenta, or by another method known in the art or described herein). In some embodiments, the CXCR3 inhibitor is administered before a diagnosis is made (and optionally continuing after the diagnosis is made), e.g., during a period of time while a blood culture assay is running (e.g., for 1 to 2 days before the results of the assay are available).

In some embodiments, the CXCR3 inhibitor is administered to the subject during or after manifestation of one or more symptoms of infection, or during or after an infection, such as within 12 or 24 hours of an infection or manifestation of one or more symptoms of infection. In some embodiments, the CXCR3 inhibitor is administered to the subject within 7 days (e.g., within 7, 6, 5, 4, 3, 2, or 1 days) after the subject is infected with the pathogen such as Listeria monocytogenes or manifests a symptom of the infection.

Methods and compositions described herein are meant for any subject that is pregnant, such as a pregnant human female subject or any other pregnant female mammal (e.g., mouse, dog, cat, rat, rabbit, horse, cow, pig, goat, or non-human primate).

A subject can be identified as pregnant using any method known in the art. Symptoms of pregnancy include nausea, vomiting, fatigue, unusual food cravings, frequent urination, and breast tenderness. Other signs and symptoms of pregnancy cessation of menstrual period, swelling of the abdomen, increased body temperature for more than 2 weeks after ovulation, Chadwick's sign (darkening of the cervix, vagina, and vulva), Goodell's sign (softening of the vaginal portion of the cervix), Hegar's sign (softening of the uterus isthmus), and pigmentation of linea alba—Linea nigra, (darkening of the skin in a midline of the abdomen)). Biomarkers for pregnancy include early pregnancy factor (EPF) and human chorionic gonadotropin (hCG), which can be detected, e.g., by a blood test or a urine test strip. Ultrasound may also be used to identify pregnancy.

In some embodiments, the subject has or is at risk of having an infection, a stillbirth, preeclampsia or a premature infant. In some embodiments, the subject is a subject that has or is at risk of having an infection such as Listeria monocytogenes In some embodiments, the subject is a pregnant individual at risk for stillbirth, which refers to the intrauterine death and subsequent delivery of a non-viable fetus after 20 gestational weeks. In some embodiments, the subject is a pregnant individual that has or is at risk of having preeclampsia. Preeclampsia refers to a pregnancy complication characterized by high blood pressure and signs of damage to another organ system, for example, the kidneys. It usually begins after 20 weeks of pregnancy in a woman whose blood pressure had been previously normal. Even a slight rise in blood pressure may be a sign of preeclampsia. In some embodiments, the subject is a subject that has or is at risk of having a premature infant.

In some embodiments, the infection is caused by a pathogen selected from the group consisting of a virus, a fungus, a parasite or a bacterium. Exemplary pathogens include Listeria monocytogenes, Influenza A, herpes simplex virus, Lymphocytic Choriomeningitis Virus (LCMV), Salmonella, Plasmodium, Toxoplasma, Escherichia coli, cytomegalovirus (CMV), parvovirus, or Leishmania spp. Infection can be detected using any method known in the art. Exemplary methods of detection include culture of blood, blood, amniotic fluid, spinal fluid or placenta to detect the pathogen, nucleic-acid based assays (e.g., PCR, reverse transcriptase-PCR, strand displacement amplification, transcription amplification, branched DNA assays, hybrid capture, ligase chain reaction, cleavase-invader, cycling probes, sequencing, microarray analysis, and melting curve analysis), serology and other immunological assays (e.g., agglutination assay, complement fixation, enzyme immunoassays, precipitation assay, Western blot) a blood smear, chromatography, mass spectrometry, gram stain, acid-fast or moderate (modified) acid-fast stains, India ink (colloidal carbon) stain, Warthin-Starry stain, Dieterle stain, Trichrome stain (Gomori-Wheatley stain), iron hematoxylin stain, or detection of the pathogen or a biomarker of the pathogen in a urine sample, a blood sample or a fecal sample (see, e.g., The Merck Manual. 19^{th} Edition. Infectious Diseases. Editor Robert S. Porter. Merck Publishing Group.).

In some embodiments, the subject has one or more symptoms of infection. Exemplary symptoms of infection include fever (e.g., ranging between 38.2° C. and 41.2° C.), abdominal pain, back pain, vomiting, diarrhea, headache, myalgia, runny nose, sinus congestion, cough, itching or burning sensation, a white blood cell count from 3900 to 33,800 cells/mm3 or sore throat. Detection of these symptoms and other symptoms are within the skill of an medical practitioner.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1. CXCR3 Blockade Protects Against Listeria monocytogenes Infection Induced Fetal Wastage

### Introduction

Stillbirth, defined as fetal loss in the second half of pregnancy, remains a pressing global health problem with devastating medical and psychosocial consequences. The World Health Organization estimates that 2.6 million cases occur annually (Kelley, Lancet 377(9778), 1636-7 (2011)). Although the etiology in most cases is multi-factorial and not definitively identified, maternal infection is undoubtedly an important causative factor (Goldenberg et al., Am. J. of Obstetrics and Gynecology 198(3), 861-73 (2003); Gibbs, Semin. Perinatol. 26(1), 75-8 (2002); Goldenberg et al., Lancet, 375(9724), 1482-90 (2010)). In this regard, while pathogens best implicated in causing stillbirth are limited to a handful of ubiquitous bacterial, viral, or parasitic microbes capable of intracellular invasion and/or genital-urinary colonization (Goldenberg et al., Am. J. of Obstetrics and Gynecology 198(3), 861-73 (2003); Gibbs, Semin. Perinatol. 26(1), 75-8 (2002); Goldenberg et al., Lancet 375(9724), 1482-90 (2010); Robbins et al., Curr. Opin. Microbiol. 15(1), 36-43 (2012); Kourtis et al., N Eng J of Med. 370(23), 2211-8 (2014)), the rapid kinetics whereby fetal injury can occur coupled with this microbial heterogeneity still preclude the use of antimicrobials targeting individual pathogens as a means of therapy or prevention. Accordingly, dissecting the underlying pathophysiology causing fetal wastage represents important perquisites for new strategies aimed at more universal protection against infection induced pregnancy complications.

Among pathogens that cause prenatal infection, the Gram-positive intracellular bacterium, Listeria monocytogenes (Lm), has unique predilection for disseminated maternal infection with often mortal consequences for the developing fetus (Silver et al., Obstet Gynecol Surv. 53(12), 737-40 (1998); Lamont et al., J Perinat Med. 39(3), 227-36 (2011)). For example, in 178 cases of prenatal Lm infection, 20% of pregnancies terminated in abortion or stillbirth, and 68% of live offspring were infected (Mylonakis et al. Medicine (Baltimore) 81(4), 260-9 (2002). This predisposition for fetal wastage and disseminated Lm infection during pregnancy is not limited to only humans, but widely reiterated across mammalian species including non-human primates (Smith et al., Infect Immun. 71(3), 1574-9 (2003)), ruminants (Linde et al., Vaccine. 13(10), 923-6 (1995); Gitter et al., Vet Rec. 118(21), 575-8 (1986)) and rodents (Bakardjiev et al., Infect Immun. 72(1), 489-97 (2004); Guleria et al., Nat Med. 6(5), 589-93 (2000); Redline et al., J Clin Invest. 79(4), 1234-41 (1987)). Recent studies using mice bearing allogeneic pregnancy designed to recapitulate the natural heterogeneity between maternal-fetal MHC haplotype antigens indicate prenatal Lm infection induced fetal resorption may not require direct in utero bacterial invasion (Rowe et al., PLoS Pathog. 8(8), e1002873 (2012)). Instead, overriding suppression by expanded maternal Foxp3+ regulatory CD4+ T cells (Tregs) by attenuated Lm that do not cross the placental-fetal barrier triggers sterile fetal wastage, along with expansion and IFN-γ production by maternal T cells with fetal specificity (Rowe et al., PLoS Pathog. 8(8), e1002873 (2012); Ertelt et al., J of Immunol. 187(5), 2569-77 (2011); Le Monnier et al., Infect Immun. 75(2), 950-7 (2007)). Direct associations between blunted maternal Treg expansion or their dampened suppressive properties are also increasingly recognized in many idiopathic pregnancy complications linked with disruptions in fetal tolerance (e.g. preeclampsia, spontaneous abortion, prematurity) (Prins et al., Hypertens Pregnancy. 28(3), 300-11 (2009); Santner-Nanan et al., J of Immunol. 183(11), 7023-30 (2009); Sasaki et al., Clin Exp Immunol. 149(1), 139-45 (2007); Sasaki et al., Mol Hum Reprod. 10(5), 347-53 (2004); Schober et al., Immunol Cell Biol. 90(10), 935-44 (2012); Jiang et al., J of Immunol. 192(11), 4949-56 (2014)). This necessity for expanded maternal Tregs modeled in animal pregnancy shows even partial transient depletion of Foxp3+ cells to pre-pregnancy levels unleashes expansion and activation of IFN-γ producing maternal CD8+ effector (Tc1) and CD4+ helper (Th1) T cells with fetal specificity that share striking commonality with disruptions in fetal tolerance instigated by prenatal Lm infection (Rowe et al., Cell Host Microbe. 10(1), 54-64 (2011); Rowe et al., Nature. 490(7418), 102-6 (2012)). Thus, overriding fetal tolerance with ensuing activation of maternal immune components with fetal specificity may play universal roles in the pathogenesis of pregnancy complications.

Limiting T cell access to the decidua in healthy pregnancy explains protection against fetal loss despite high circulating levels of activated maternal T cells with defined fetal specificity (Nancy et al., Science. 336(6086), 1317-21 (2012); Perchellet et al., Biol of Reproduction. 89(4), 102 (2013)).

Herein, the immune-pathogenesis of fetal injury triggered by infectious and non-infectious disruptions in fetal tolerance was investigated using mouse pregnancy where ovalbumin is transformed into a surrogate fetal antigen. Prenatal Lm infection was found to unleash recruitment of Th1/Tc1 chemokine producing inflammatory cells to the decidua, circumventing the normally protective immunological barrier restricting fetal-specific T cells from the maternal-fetal interface.

The present study indicated that neutralizing the receptor for Th1/Tc1 inducing chemokines, CXCR3, before or shortly after prenatal Lm infection efficiently protects against fetal wastage. The protective benefits conferred by CXCR3 blockade extend to immune-mediated fetal wastage induced by intrapartum depletion of maternal Tregs. Thus, dissecting the underlying immune pathogenesis of prenatal infection reveal chemokine receptors such as CXCR3 as new therapeutic targets for averting pregnancy complications and preventing stillbirth. Blocking the CXCR3 signaling may inhibit maternal Th1/Tc1 effector T cells and maintain fetal tolerance in the presence of either prenatal infectious or non-infectious disruptions, thereby protecting fetus against unintentional attack by maternal immune components.

### Materials and Methods

Mice.

C57BL/6 (B6, H-2^{b}), Balb/c (H-2^{d}), and Rag2-deficient mice on the B6 background were purchased from the National Cancer Institute (Bethesda Md.) or Jackson Laboratory (Bar Harbor Me.). The use of transgenic mice that ubiquitously express OVA protein behind the constitutively active β-actin promoter to sire pregnancy in non-transgenic females transforming OVA into a surrogate fetal antigen has been described (Perchellet et al., Biol of Reproduction. 89(4), 102 (2013); Ehst et al., Am J Transplant. 3(11), 1355-62 (2003); Erlebacher et al., J of Clin Investig. 117(5), 1399-411 (2007)). OVA-expressing mice were backcrossed >10 generations to Balb/c mice in the studies to establish allogeneic pregnancy (Rowe et al., PLoS Pathog. 8(8), e1002873 (2012); Rowe et al., Cell Host Microbe. 10(1), 54-64 (2011)). P14 TCR transgenic mice where all CD8⁺ T cells have exclusive monoclonal specificity to LCMV-GP₃₃₋₄₁ peptide (Pircher et al., Nature. 342(6249), 559-61 (1989)) on a Rag2-deficient B6 background were purchased from Taconic farms (Cambridge City Ind.). OT-1 TCR transgenic mice where all CD8⁺ T cells have exclusive monoclonal specificity to OVA₂₅₇₋₂₆₄ peptide (Hogquist et al., Cell. 76(1), 17-27 (1994)), were maintained on a Rag2-deficient CD90.1 congenic background. Foxp3^{DTR} and the use of Foxp3^{DTR/WT} female mice to investigate the necessity for sustained maternal Treg expansion during allogeneic pregnancy have been described (Rowe et al., Cell Host Microbe. 10(1), 54-64 (2011); Rowe et al., Nature. 490(7418), 102-625 (2012); Kim et al., Nat Immunol. 8(2), 191-7 (2007); Samstein et al., Cell. 150(1), 29-38 (2012)). For partial transient depletion of maternal Tregs, Foxp3^{DTR/WT} females sired by Balb/c males were administered purified DT (Sigma-Aldrich, St. Louis Mo.) daily (0.5 μg first dose, followed by 0.1 μg/dose thereafter) beginning midgestation (E11.5) for five consecutive days, and frequency of fetal resorption evaluated E16.5 as previously described (25, 26). Where indicated, pregnant mice were administered autoclaved drinking water supplemented with ampicillin (0.5 mg/ml) (Sigma-Aldrich).

Listeria monocytogenes and influenza A infections. Wildtype virulent Lm strain 10403s, the isogenic ΔactA attenuated strain DPL1942, and recombinant ΔactA Lm-OVA have each been described (Rowe et al., PLOS Pathol. 8(8), d1002873 2012; Way et al., J of Immunol. 178(7), 4498-505 (2007); Foulds et al. J of Immunol. 168(4), 1528-32 (2012); O'Riordan et al., PNAS. 99(21):13861-6 (2002)). For infection, Lm were grown to early log phase (OD₆₀₀ 0.1) in brain heart infusion media at 37° C., washed and diluted with saline to 200 μl, and injected via the lateral tail vein at the following dosages (Lm ΔactA, 10⁷ CFUs; Lm 10403s, 10⁴ CFUs) as described (Rowe et al., PLoS Pathog. 8(8), e1002873 (2012)). For each infection, the inoculum was verified by plating serial dilutions onto agar media. For enumerating recoverable Lm CFUs, individual concepti (placentae and feti), along with spleen, liver, lung, kidney and brain were sterilely dissected, homogenized in saline containing 0.05% Triton X and spread onto agar plates as described (Rowe et al., PLoS Pathog. 8(8), e1002873 (2012); Rowe et al., Cell Host Microbe. 10(1), 54-64 (2011)). Influenza A virus serotype H1N1 strain PR8 has been described (Palese et al., PNAS, 73(6), 2142-6 (1976)). For infection, mice were anesthetized with ketamine and xylazine, and inoculated intranasally with 10³ PFU suspended in 30 μl sterile saline. Five days after infection, viral recovery was evaluated by plating serial dilutions of each organ homogenate onto Madin-Darby canine kidney cells, and enumerating plaques 18 hours thereafter (Tobita et al., Med Microbiol and Immunol. 162(1), 23-7 (1975)).

Tissue Harvest, Antibodies, and Flow Cytometry.

Single cell suspensions of splenocytes and paraaortic lymph node cells were prepared by gentle tissue dissociation between frosted glass slides, and RBC lysis in ammonium chloride buffer. Decidua and myometrium were isolated as previously described (Collins et al., J Clin Invest. 119(7), 2062-73 (2009)). Specifically for flow cytometry, uteri were dissected in ice-cold Hanks Balance Salt Solution (HBSS), and the myometrium was peeled away from each individual concepti and placentae. Placentae were then removed from the feti and separated at the interface of the labyrinth and junctional zone for analysis of the decidua (decidua basalis and junctional layers), placed in RBC lysis buffer, and dissociated into single cell suspension by grinding between frosted glass slides. Thereafter, DMEM was added to quench the lysis reaction, samples were filtered through a 70 μm cell strainer and pelleted by centrifugation (1600 rpm for 5 min). The tissue cell pellets were then suspended in DMEM media supplemented with 10% fetal bovine serum, and used for cell surface and intracellular staining. For histological analysis, the uterine wall and placenta were harvested en bloc and fixed in 10% paraformaldehyde (4° C. for 12-16 hours). Thereafter, the tissue was embedded in paraffin, cut into 5 μm sections, deparaffinized in xylene and rehydrated with ethanol. Endogenous fluorescence was reduced using 0.1% sodium borohydride. Non-specific protein interactions were blocked using goat serum (5%) and bovine serum albumin (1%). Sections were then incubated with PE conjugated CD90.1 (clone OX-7) for 1 hour at room temperature, and mounted with Vectashield media with DAPI (Vector laboratories, Burlingame Calif.). For cytokine production, cells were stimulated with PMA/ionomycin for 5 hours in media supplemented with brefeldin A (GolgiPlug, BD Biosciences, San Jose Calif.) prior to cell surface and intracellular staining. For CXCL9 expression, cells recovered from the decidua or myometrium were incubated in media supplemented with brefeldin A for 5 hours followed by cell surface and intracellular staining using anti-CXCL9 antibody (2F5.5). Other fluorophore-conjugated antibodies including anti-CD4 (GK1.5 and RM4-4), anti-CD8α (53-6.7), anti-CD8β (H35-17.2), anti-NK1.1 (PK136), anti-CD90.1 (HIS51), anti-CD45.1 (A20), anti-45.2 (104), anti-CD11b (M1/70), anti-CD11c (N418), anti-B220 (RA3-B62), anti-F4/80 (BM8), anti-CXCR3 (CXCR3-173), and IFN-γ (XMG1.2) used for cell surface and intracellular staining were purchased from eBioscience (San Diego Calif.) or BD Biosciences. Purified antibodies for in vivo T cell depletion (anti-CD4, GK1.5; anti-CD8, 2.43), IFN-γ neutralization (XMG1.2), neutrophil and macrophage cell depletion (anti-Gr1, RB6-8C5), CXCR3 neutralization (CXCR3-173), along with rat and hamster IgG isotype control antibodies were purchased from BioXcell (West Lebanon N.H.) and administered intraperitoneally (500 μg each antibody per mouse).

Adoptive Cell Transfers.

OVA-specific CD8⁺ T cells were isolated from CD90.1⁺ congenic OT-I mice, and injected intravenously into CD90.2⁺ recipients (10⁵ donor OT-1 CD8⁺ T cells) one day prior to Lm infection or DT administration (to Foxp3^{DTR/WT} mice) at midgestation (E11.5). Thereafter, accumulation of OVA-specific T cells in each tissue was evaluated by gating on CD90.1⁺ donor among CD90.2 recipient cells as described (Rowe et al., PLoS Pathog. 8(8), e1002873 (2012); Rowe et al., Cell Host Microbe. 10(1), 54-64 (2011)).

Intravascular Staining.

To discriminate between tissue resident and intravascular leukocytes, staining with fluorochrome conjugated anti-CD45.2 antibody after intravenous injection immediately prior to euthanasia was performed as described previously (Anderson et al., Nat Protoc. 9(1), 209-22 (2014)). Specifically, 3 μg of PE conjugated anti-CD45.2 antibody (clone 104) was injected through the lateral tail vein three minutes prior to euthanasia and tissue harvest.

Statistics.

Differences in fetal resorption frequency, number of live pups, percent infected concepti, and cell numbers were analyzed using the unpaired Student t test with P<0.05 taken as statistical significance.

### Results

### Maternal CD8⁺ T Cells and IFN-γ are Essential for Prenatal Lm Infection Induced Fetal Wastage

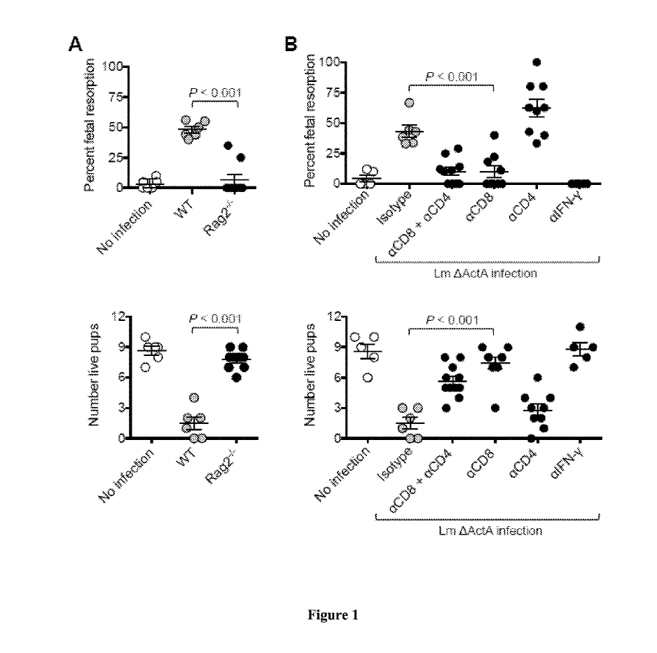

To investigate whether maternal adaptive immune components are essential for infection induced fetal wastage, pregnancy outcomes were evaluated in Rag2-deficient mice completely lacking T and B cells after Lm prenatal infection initiated midgestation (E11.5) during allogeneic pregnancy. To bypass infection susceptibility in the absence of ‘innate’ T cells (Berg et al., Eur J of Immunol. 32(10), 2807-16 (2002); O'Donnell et al., Immunity. 40(2), 213-24 (2014)), an attenuated ΔactA Lm strain that cannot cause productive infection due to defects in intercellular spread while still retaining the ability to fracture fetal tolerance and induce sterile fetal resorption was utilized (Rowe et al., PLoS Pathog. 8(8), e1002873 (2012); Le Monnier et al., Infect Immun. 75(2), 950-7 (2007)). Fetal resorption with loss of live pups induced by Lm ΔactA prenatal infection among immune-competent C57BL/6 mice was found to be eliminated in isogenic Rag2-deficient mice to background levels found in uninfected control pregnancies (FIG. 1A). Thus, maternal adaptive immune components are essential for Lm infection induced fetal wastage.

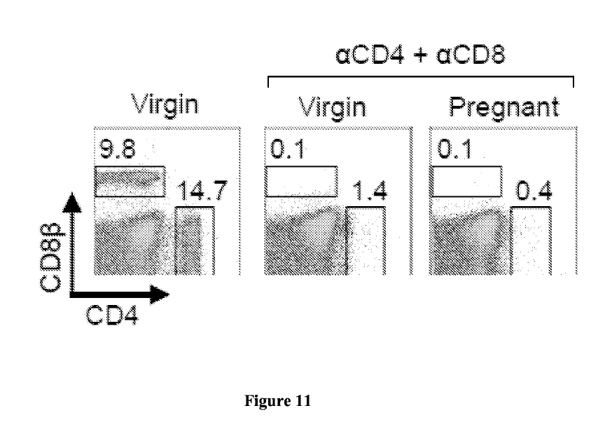

Considering prenatal infection with virulent or ΔactA attenuated Lm each prime expansion of maternal T cells with fetal specificity (Rowe et al., PLoS Pathog. 8(8), e1002873 (2012)), pregnancy outcomes were evaluated after depletion of CD4⁺ and CD8⁺ T cells individually or concurrently one day prior to Lm inoculation to more specifically investigate the necessity for each T cell subset in infection induced fetal wastage. Pregnancy did not impact the efficiency of T cell depletion using well characterized monoclonal antibodies (Johanns et al., Microbes and infection/Institut Pasteur. 13(4), 322-30 (2011)), and the frequency of fetal resorption and loss of live pups were sharply reduced with depletion of both CD4⁺ and CD8⁺ T cells prior to Lm infection in line with protection from fetal wastage in Rag2-deficient mice (FIGS. 1 and 11). Depletion of CD8⁺ T cells alone conferred similar protective benefits with fetal resorption and loss of live pups reduced to background levels comparable to uninfected control mice (FIG. 1B). By contrast, fetal resorption and loss of live pups increased significantly when CD4⁺ T cells were depleted in isolation, consistent with disruptions in fetal tolerance that occur after eliminating immune suppressive Tregs restricted to the CD4⁺ subset of peripheral T cells (FIG. 1B) (Fontenot et al., Nat Immunol. 4(4), 330-6 (2003); Hori et al., Science. 299(5609), 1057-61 (2003)). To further investigate the necessity for IFN-γ cytokine produced by maternal CD8⁺ T cells primed by prenatal Lm infection (Rowe et al., PLoS Pathog. 8(8), e1002873 (2012)), pregnancy outcomes were evaluated in mice treated with anti-IFN-γ neutralizing antibody. These experiments showed IFN-γ neutralization also efficiently reversed Lm ΔactA induced fetal wastage as only background levels of fetal resorption with negligible loss of live pups were observed (FIG. 1B). Collectively, these results indicate maternal CD8⁺ T cells and IFN-γ are each essential for fetal wastage triggered by prenatal ΔactA Lm infection.

Infection Induced Fetal Wastage Requires Maternal CD8⁺ T Cells with Fetal Specificity

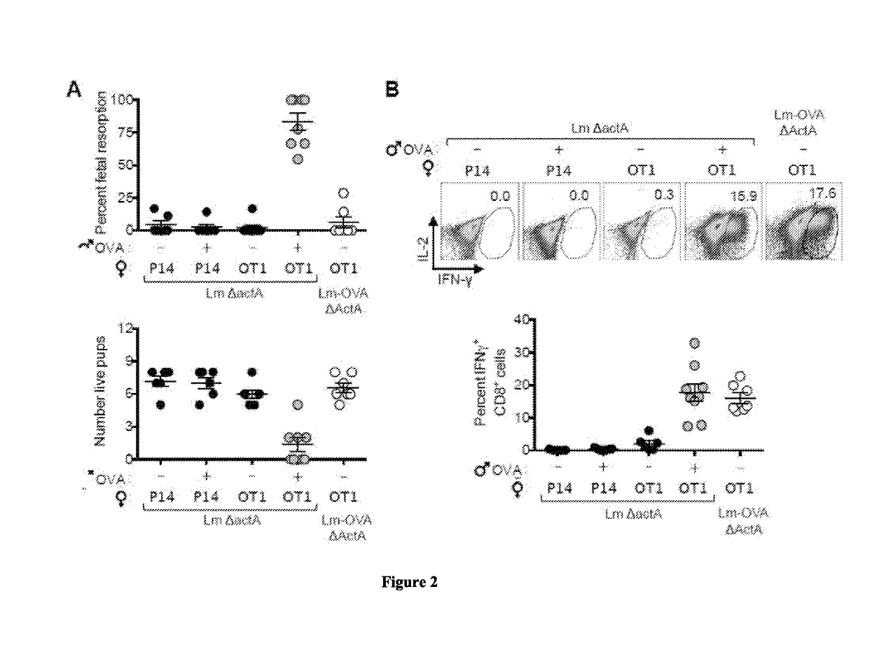

To address whether maternal CD8⁺ T cells with fetal specificity drive fetal wastage after prenatal infection, pregnancy outcomes were evaluated among T cell receptor (TCR) transgenic mice containing CD8⁺ T cells with fixed monoclonal specificity. In particular, P14 and OT-1 TCR transgenic mice with exclusive CD8⁺ T cell specificity to MHC class I restricted lymphocytic choriomeningitis virus (LCMV)-GP₃₃₋₄₁ and ovalbumin (OVA)₂₅₇₋₂₆₄ peptides, respectively (Pircher et al., Nature. 342(6249), 559-61 (1989); Hogquist et al., Cell. 76(1), 17-27 (1994)), were maintained on a Rag2-deficient background to exclude potential recognition of other antigens through dual TCR expression (Manning et al., J of Immunol. 159(10), 4665-75 (1997)), and reconstituted with bulk splenocytes from CD8α-deficient mice to restore a polyclonal repertoire of CD4⁺ T and B cells. Using these mice with fixed monoclonal CD8⁺ T cell specificity, Lm infection induced fetal wastage and IFN-γ production by maternal CD8⁺ T cells were each found to be eliminated if maternal CD8⁺ T cells did not have overlapping fetal specificity (pregnancy in P14 female mice containing CD8⁺ T cells with LCMV-GP₃₃₋₄₁ specificity sired by Balb/c males or transgenic male mice engineered to constitutively express OVA as a trans-membrane protein ubiquitously in all cells behind the β-actin promoter on the Balb/c background [Balb/c-OVA] (Rowe et al., Cell Host Microbe. 10(1), 54-64 (2011); Ehst et al., Am J Transplant. 3(11), 1355-62 (2003)); or pregnancy in OT-1 female mice containing CD8⁺ T cells with OVA₂₅₇₋₂₆₄ specificity sired by Balb/c males) (FIG. 2). On the other hand, fetal resorption, loss of live pups, and IFN-γ production by maternal CD8⁺ T cells each rebounded when maternal CD8⁺ T cell specificity was engineered to overlap with fetal expressed antigen (pregnancy in OT-1 female mice containing maternal CD8⁺ T cells with OVA₂₅₇₋₂₆₄ specificity sired by Balb/c-OVA males) (FIG. 2). To further investigate whether protection from fetal wastage in pregnancies without overlap in specificity between maternal CD8⁺ T cells and fetal antigen could be explained by less activated CD8⁺ T cells, pregnancy outcomes were enumerated in OT-1 female mice after infection with recombinant Lm expressing OVA as a pathogen-associated antigen that primes expansion of IFN-γ producing OVA-specific CD8⁺ T cells (Foulds et al., J of Immunol. 168(4), 1528-32 (2012); Way et al., J of Immunol. 178(7), 4498-505 (2007)). During allogeneic pregnancy sired by non-transgenic Balb/c males, fetal wastage remained at background levels despite robust IFN-γ production by activated maternal CD8⁺ T cells with non-fetal OVA specificity (FIG. 2). Thus, maternal CD8⁺ T cell recognition of fetal antigen is essential for infection induced fetal injury, whereas non-specific activation of maternal CD8⁺ T cells in isolation is insufficient to cause fetal wastage.

Decidual Accumulation of Maternal CD8⁺ T Cells with Fetal Specificity after Prenatal Lm Infection

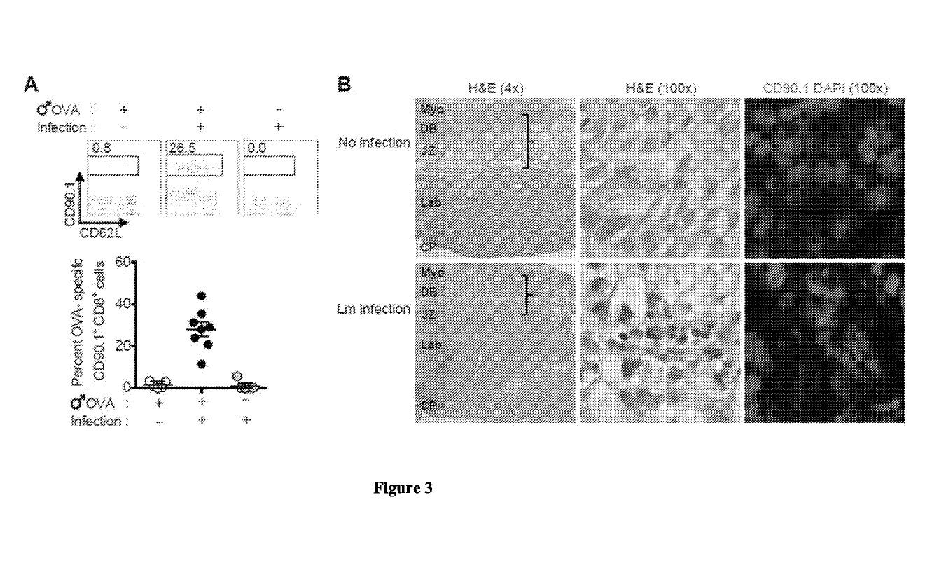

The potential for decidual accumulation of fetal-specific CD8⁺ T cells after prenatal Lm infection was investigated as follows. To more precisely identify maternal T cells with fetal specificity, OVA₂₅₇₋₂₆₄-specific CD8⁺ T cells from the aforementioned OT-1 TCR transgenic mice maintained on the CD90.1 congenic background were tracked after adoptive transfer into CD90.2⁺ female recipients during allogeneic pregnancy sired by Balb/c-OVA males (Rowe et al., PLoS Pathog. 8(8), e1002873 (2012); Rowe et al., Cell Host Microbe. 10(1), 54-64 (2011); Erlebacher et al., J Clin Invest. 117(5), 1399-411 (2007)). CD8⁺ T cells with surrogate fetal-OVA specificity (identified by expression of the CD90.1 congenic marker) were found to accumulate by >20-fold in the decidua compared with background levels in uninfected control mice within the first three days after prenatal Lm ΔactA infection (FIG. 3A). A slightly earlier post-infection time point was utilized to bypass inconsistent recovery of decidual tissue that occurs with fetal wastage five days after infection (Clark et al., Cellular and Mol Immunol. 11(6), 595-605 (2014)). Decidual accumulation of maternal CD8⁺ T cells reflects a targeted response to fetal antigen stimulation because OVA-specific CD90.1⁺ CD8⁺ T cells remained at background levels after Lm ΔactA infection in pregnant mice sired by Balb/c (non-OVA expressing) males (FIG. 3A). Infection induced accumulation of maternal CD8⁺ T cells with fetal-OVA-specificity in the placenta was verified by immunohistochemistry using anti-CD90.1 antibodies. Along with diffuse infiltration of mixed polymorphonuclear and lymphocytic inflammatory cells, patches of fetal-OVA-specific CD90.1⁺ cells were scattered throughout the decidua basalis and junctional zone selectively for OVA⁺ concepti after prenatal Lm infection, whereas CD90.1⁺ staining cells were absent in both sham-infected controls or OVA⁻ concepti regardless of infection (FIG. 3B and data not shown).

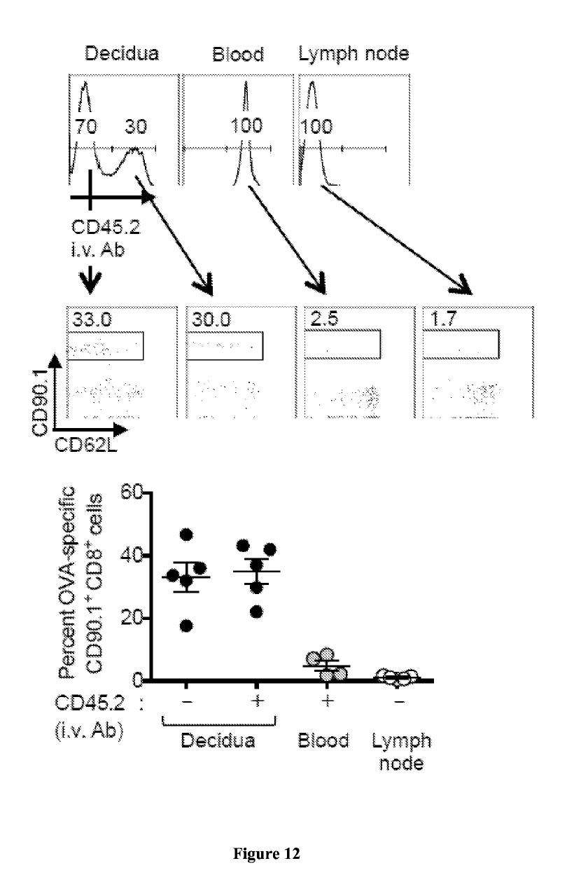

To further investigate the possibility that maternal T cells enriched within highly vascular decidual tissue could reflect contamination by intravascular cells, staining with fluorochrome-labeled antibody against the pan-leukocyte CD45 antigen injected intravenously immediately prior to euthanasia was evaluated (Anderson et al., Nat Protoc. 9(1), 209-2242 (2014)). This analysis showed that while cells recovered from the blood uniformly stained positive and paraaortic lymph node cells consistently did not bind intravenously injected α-CD45.2 antibody (positive and negative controls), ˜70% of CD8⁺ T cells recovered from the decidua also did not bind intravenously injected antibody suggesting this cellular majority is of non-vascular origin (FIG. 12). Regardless of staining with intravenously injected α-CD45.2 antibody, CD90.1⁺ cells with surrogate fetal-OVA specificity were equally enriched among CD8⁺ T cells recovered from the decidua compared with blood or lymph node cells demonstrating massive enrichment of fetal-OVA specific CD8⁺ T cells among both tissue resident and intravascular decidual cells. Together, these results show prenatal Lm infection induced fetal wastage parallels robust accumulation of maternal CD8⁺ T cells with fetal specificity at the maternal-fetal interface.

Decidual Infiltration of CXCL9-Expressing Leukocytes and CXCR3-Expressing CD8⁺ T Cells after Prenatal Lm Infection

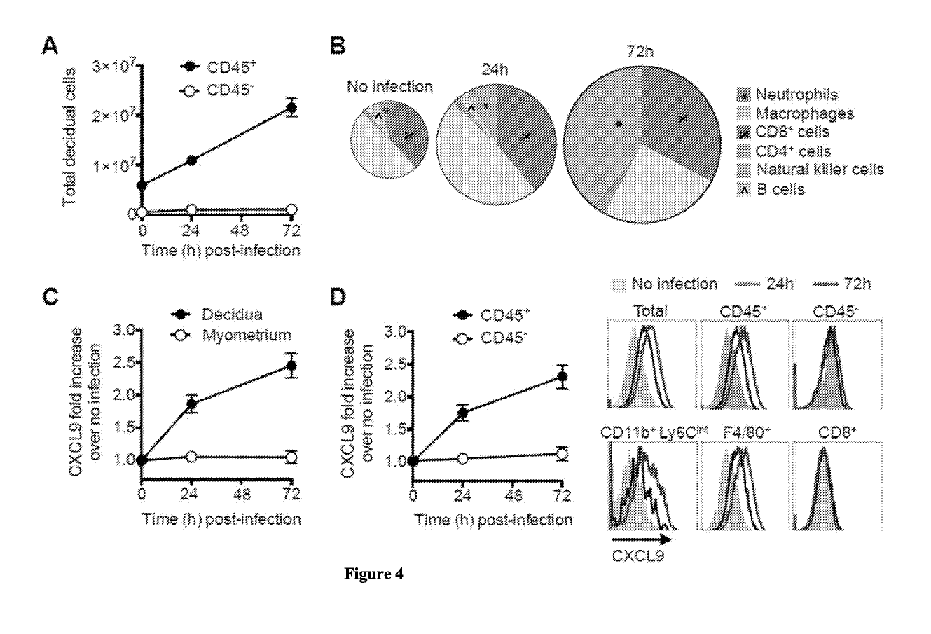

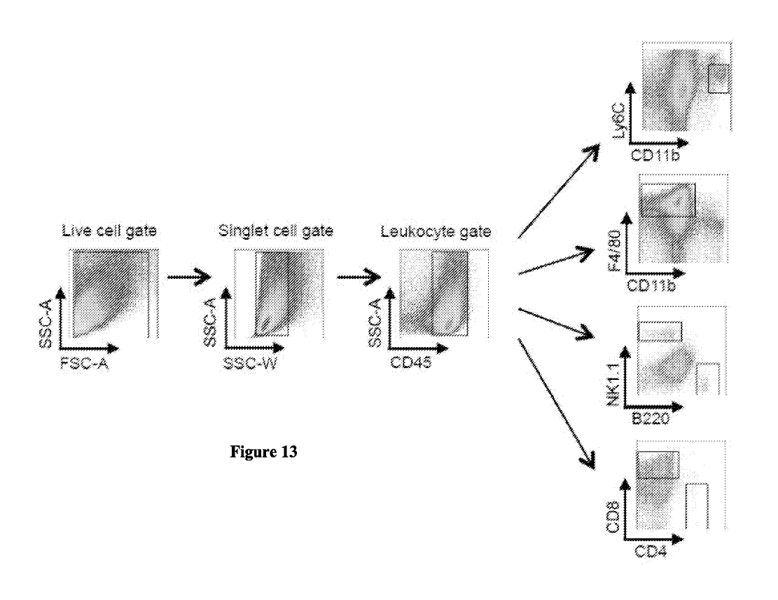

To further define shifts in cells that infiltrate the decidua after prenatal Lm infection, the number and composition of cells in this tissue was evaluated. Within the first 24 hours after Lm ΔactA inoculation, the absolute number of decidual cells increased by ˜2-fold comprised almost exclusively by CD45⁺ leukocytes with composition similar to uninfected control mice (FIGS. 4A, 4B, and 13). As infection progressed, quantitative as well as qualitative shifts in CD45⁺ decidual leukocytes were observed reaching ˜4-fold expanded levels with sharply enriched proportions of CD11b⁺ Ly6C^{int} neutrophils by 72 hours after Lm ΔactA infection (FIGS. 4A and 4B). Thus, decidual accumulation of maternal CD8⁺ T cell with fetal specificity after prenatal Lm infection parallels the influx of neutrophils and other leukocyte subsets to the maternal-fetal interface.

It was further investigated whether decidual accumulation of fetal-specific CD8⁺ T cells triggered by prenatal Lm infection circumvents chemokine expression silencing. Within the first 72 hours after Lm ΔactA infection, expression of the prototypical Th1/Tc1 chemokine, CXCL9 increased progressively among cells recovered from the decidua compared with the adjacent myometrium (FIG. 4C). However, CXCL9 expression among decidual cells was comprised almost exclusively by CD11b⁺ Ly6C^{int} neutrophils and F4/80⁺ macrophage cells, whereas CXCL9 expression remained at background levels among CD45⁻ stromal cells and CD8⁺ T cells (FIG. 4D).

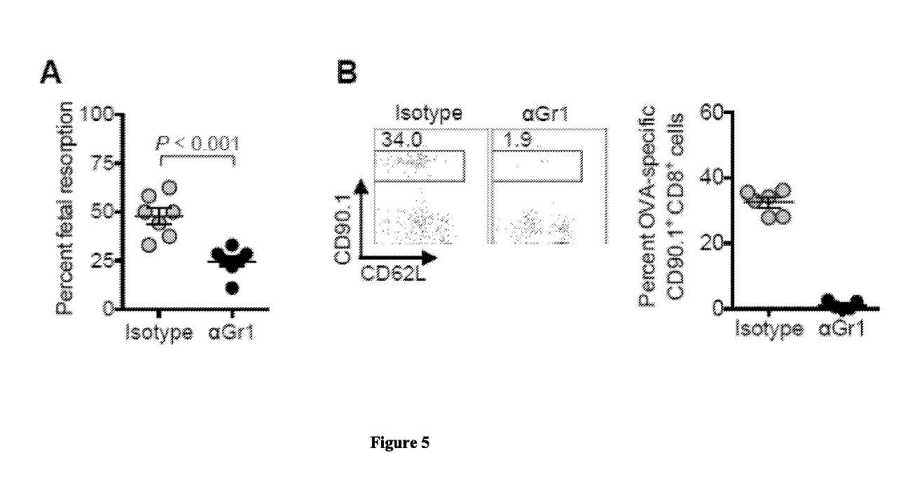

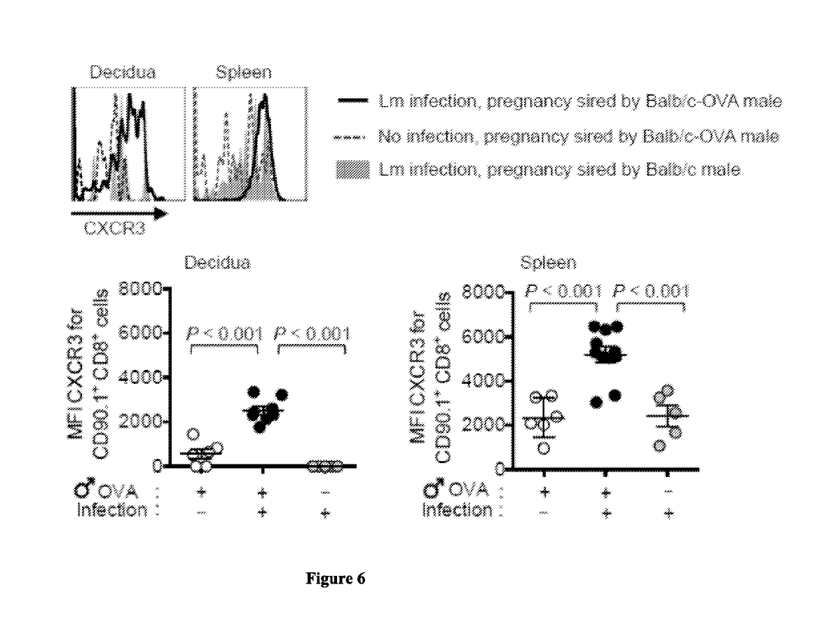

To address the necessity for these specific CXCL9 producing cell subsets in infection induced fetal wastage, the impacts of their in vivo depletion on fetal resorption and decidual infiltration by CD8⁺ T cells with fetal specificity were evaluated. Administration of anti-Gr1 antibody (RB6-8C5) (Shi et al., J of Immunol. 187(10), 5293-8 (2011)), that depletes both neutrophils and inflammatory macrophage cells one day prior to Lm infection was found to significantly diminish the frequency of fetal resorption and decidual accumulation of maternal CD8⁺ T cells with surrogate fetal-OVA specificity (FIG. 5). In turn, expression of the CXCL9 receptor, CXCR3, was also sharply upregulated among fetal-OVA specific CD8⁺ T cells recovered from both the decidua and periphery (FIG. 6). Taken together, these results show the immunological barrier enforced in healthy pregnancy through Th1/Tc1 chemokine expression silencing is circumvented by Lm prenatal infection that drives influx of chemokine producing inflammatory cells and maternal CD8⁺ T cells with fetal specificity to the maternal-fetal interface.

### CXCR3 Deprivation Protects Against Lm Infection Induced Fetal Wastage

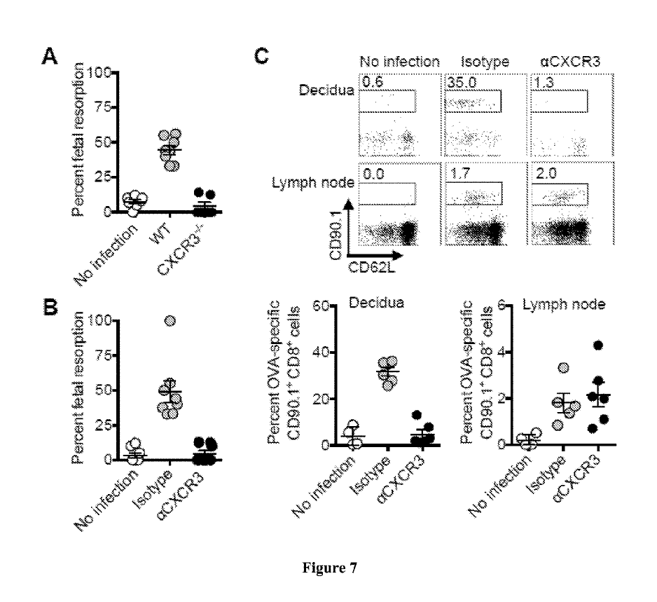

Considering multiple Th1/Tc1 inducing chemokines including CXCL9, CXCL10, CXCL11 share the common receptor, CXCR3 (Bromley et al., Nat Immunol. 9(9), 970-80 (2008)), complementary loss of function studies targeting CXCR3 were performed to more definitively establish the necessity for decidual T cell infiltration in the pathogenesis of prenatal infection. For either CXCR3-deficient mice or mice treated with CXCR3 neutralizing antibody prior to prenatal infection, fetal wastage triggered by Lm ΔactA was reduced to background levels compared with each group of CXCR3 replete control mice (FIGS. 7A and 7B). Decidual accumulation of maternal CD8⁺ T cells with fetal-OVA specificity for mice bearing allogeneic pregnancy sired by Balb/c-OVA expressing males was similarly averted by in vivo CXCR3 neutralization (FIG. 7C). Sharply reduced decidual fetal-specific CD8⁺ T cell accumulation with CXCR3 deprivation cannot be explained by diminished priming of maternal CD8⁺ T cells with fetal-OVA specificity as these cells were found in similar proportions in the paraaortic lymph node draining the uterus for anti-CXCR3 compared with isotype control antibody treated mice (FIG. 7C). Thus, despite non-essential roles for priming systemic expansion of maternal CD8⁺ T cells with fetal specificity, CXCR3 chemokine receptor stimulation is required for ΔactA Lm infection induced fetal wastage and decidual infiltration of these deleterious cells.

### CXCR3 Neutralization Protects Against Fetal Wastage Triggered by Virulent Lm Prenatal Infection

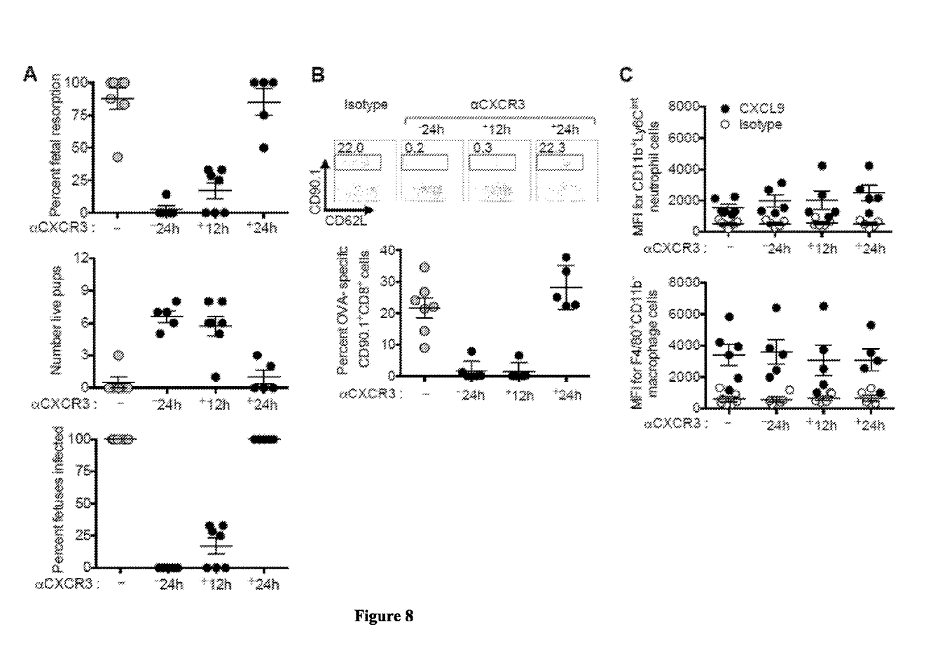

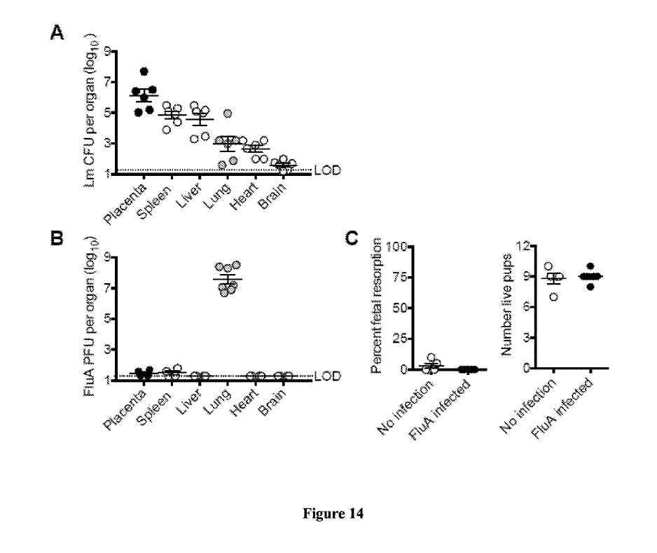

Given the efficiency whereby CXCR3 blockade protects against attenuated ΔactA Lm induced fetal resorption, the potential protective benefits of CXCR3 neutralization on fetal wastage and in utero fetal invasion after virulent Lm prenatal infection was evaluated. While systemic expansion of immune suppressive maternal Tregs required for sustaining fetal tolerance confers susceptibility to disseminated Lm infection (Rowe et al., Cell Host Microbe. 10(1), 54-64 (2011)), placental-fetal tropism appears to be equally decisive since other pathogens with more restricted tissue distribution (e.g. influenza A) that prime robust antigen-specific CD8⁺ T cell expansion do not cause fetal wastage even with high dosage maternal infection during allogeneic pregnancy (FIG. 14) (Irving et al., BJOG: an international journal of obstetrics and gynaecology. 107(10), 1282-9 (2000); Ramphal et al., Am J Obstet Gynecol. 138(3), 347-8 (1980); Tripp et al, J Immunol. 154(11), 6013-21 (1995); Gianfrani et al., Hum Immunol. 61(5), 438-52 (2000)). Accordingly, a relatively high dosage of virulent Lm (10⁴ CFUs strain 10403s) representing only a half-log₁₀ reduction from the LD₅₀ for mice bearing allogeneic pregnancy shown previously to cause consistent fetal resorption with in utero bacterial invasion (Rowe et al., PLoS Pathog. 8(8), e1002873 (2012); Rowe et al., Cell Host Microbe. 10(1), 54-64 (2011)) was utilized to investigate the impacts of CXCR3 deprivation. CXCR3 neutralizing compared with isotype control antibody administered prior to virulent Lm infection midgestation efficiently mitigated fetal resorption, loss of live pups, and bacterial invasion into individual concepti (FIG. 8A). Protection against fetal wastage paralleled near complete elimination of fetal-OVA specific CD8⁺ T cell accumulation in the decidua without significant shifts in augmented CXCL9 expression by CD11b⁺ Ly6C^{int} neutrophil and F4/80⁺ macrophage cells (FIGS. 8B and 8C). Together, these results suggest the detrimental properties of fetal-specific CD8⁺ T cell access to the decidua shown for attenuated ΔactA Lm extend to fetal wastage triggered by virulent Lm prenatal infection.

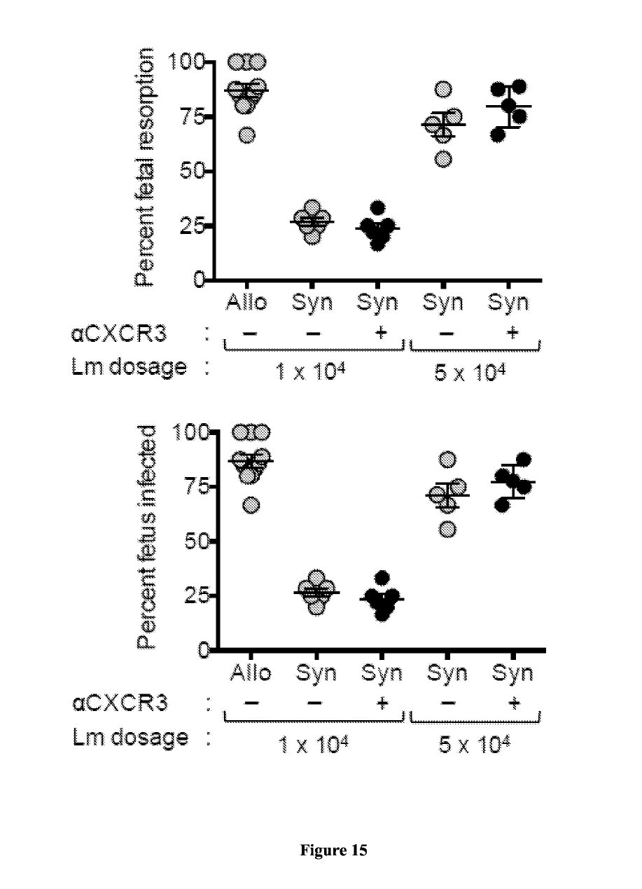

The necessity for fetal-specific CD8⁺ T cells in causing fetal wastage after virulent Lm infection was further addressed in complementary studies using mice bearing syngeneic pregnancies (C57BL/6 males mated with isogenic females). It was thought that, if activated maternal CD8⁺ T cells with specificity to immunologically discordant paternal antigen are expressed by the developing fetus, and their infiltration into the decidua drive the immune pathogenesis of prenatal infection induced fetal wastage, artificially eliminating discordance between maternal-paternal (fetal) MHC haplotype antigens in syngeneic matings would override the protective impacts of CXCR3 neutralization. In agreement with this hypothesis, susceptibility to Lm prenatal infection with regards to fetal resorption and in utero invasion frequency was significantly reduced in mice bearing syngeneic compared with allogeneic pregnancy, whereas CXCR3 blockade had no significant impacts on diminished rates of fetal wastage during syngeneic pregnancy (FIG. 15). Importantly, the ineffectiveness of CXCR3 neutralization in syngeneic compared with allogeneic pregnancies cannot be explained by diminished susceptibility to Lm infection induced fetal wastage because CXCR3 blockade also showed no protective benefits despite more uniform fetal wastage and in utero bacterial invasion achieved by increasing the dosage of virulent Lm (5-fold) used for infection during syngeneic pregnancy (FIG. 15). Thus, in the more physiological context of discordance between maternal-fetal MHC haplotype antigens recapitulated in mouse allogeneic pregnancy, preventing decidual accumulation of maternal CD8⁺ T cells with foreign fetal specificity by CXCR3 neutralization extends to protection against fetal resorption and in utero pathogen invasion after virulent Lm prenatal infection.

To further address if protection against fetal wastage shown for CXCR3 neutralization beginning prior to infection extends to ongoing prenatal infection, pregnancy outcomes were evaluated after initiating CXCR3 blockade 12 and 24 hours after virulent Lm inoculation at midgestation. Near complete protection with regards to fetal resorption, number of live pups, in utero Lm fetal invasion frequency, as well as decidual accumulation of maternal CD8⁺ T cells with fetal-OVA specificity were each only slightly diminished with initiating anti-CXCR3 antibody 12 hours post-infection compared with CXCR3 blockade before infection (FIGS. 8A and 8B). On the other hand, these protective properties were eliminated if CXCR3 blockade was delayed until 24 hours post-infection as fetal resorption, loss of live pups, Lm fetal invasion, along with accumulation of fetal-specific CD8⁺ T cells in the decidua each rebounded to levels indistinguishable from control mice without anti-CXCR3 antibody treatment (FIGS. 8A and 8B).

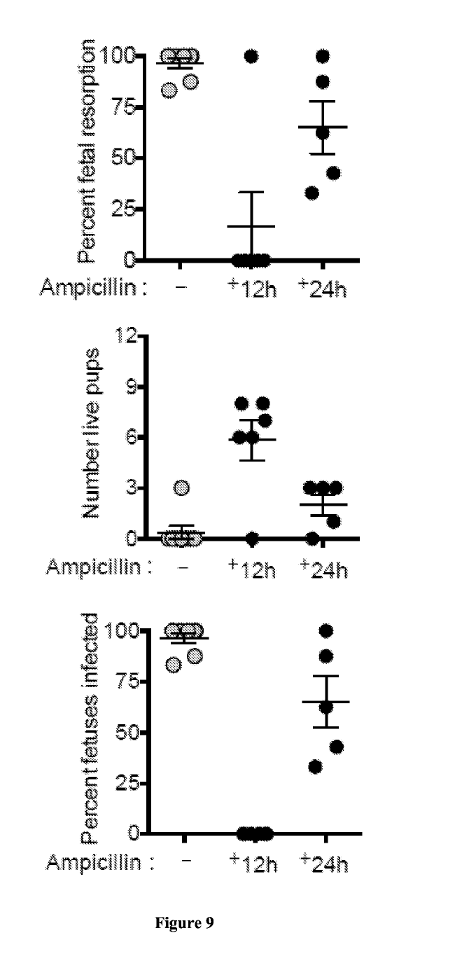

To extrapolate how these time points in mouse pregnancy may apply to a potential therapeutic window after infection for intervention in human pregnancy, the impact of delayed initiation of the current clinical gold standard therapy for Lm infection (ampicillin) on pregnancy outcomes after prenatal Lm infection was investigated (Silver, Obstet Gynecol Surv. 53(12), 737-407 (1998); Fleming et al., Obstet and Gynecol. 66(3 Suppl), 52S-3S (1985); Kalstone, Am J Obstet Gynecol. 164(1 Pt 1), 57-8 (1991)). This analysis showed protection against fetal wastage achieved with ampicillin occur with similar efficacy compared with CXCR3 neutralization at each time point after prenatal Lm infection—both convey near complete protection when initiated 12 hours after infection, whereas benefits are uniformly dissipated if delayed until 24 hours post-infection (FIGS. 8 and 9). Together, these results demonstrate CXCR3 blockade beginning prior to infection is highly efficacious in averting negative sequelae of prenatal infection, whereas initiating CXCR3 blockade shortly after infection shows therapeutic benefits comparable to the current gold standard clinical antimicrobial therapy for prenatal listeriosis.

### CXCR3 Neutralization Protects Against Fetal Resorption Triggered by Maternal Foxp3⁺ Cell Depletion

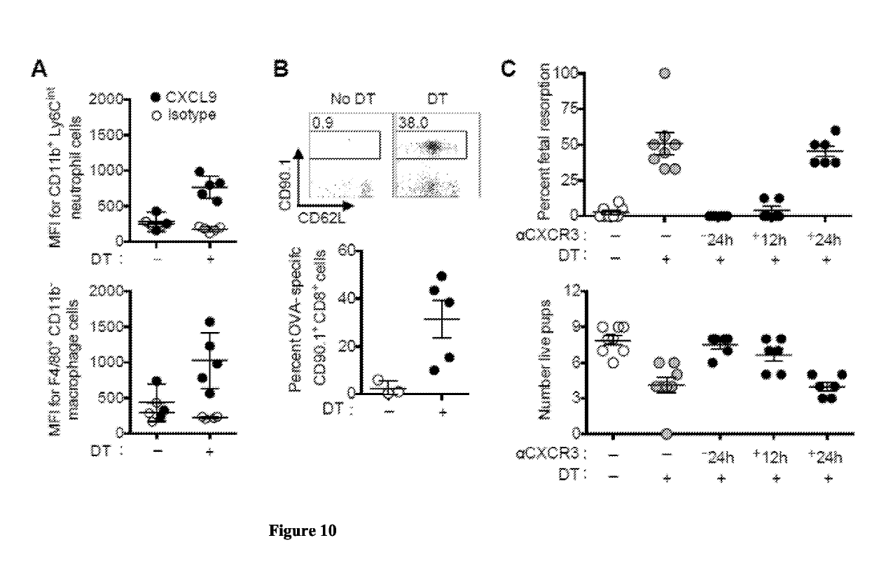

Given the potentially shared pathophysiology between fetal wastage induced by prenatal infection and non-infectious etiologies that blunt expansion of immune suppressive maternal Tregs and fracture fetal tolerance (Rowe et al., PLoS Pathog. 8(8), e1002873 (2012); Rowe et al., Cell Host Microbe. 10(1), 54-64 (2011); Rowe et al., Reproduction. 146(6), R191-203 (2013)), related studies addressed whether the protective benefits conferred by CXCR3 blockade shown for prenatal Lm infection extend to non-infectious disruptions in fetal tolerance. To mimic blunted expansion of maternal Foxp3⁺ Tregs increasingly linked with human pregnancy complications (Prins et al., Hypertens Pregnancy. 28(3), 300-11 (2009); Santner-Nanan et al., J of Immunol. 183(11), 7023-30 (2009); Sasaki et al., Clin Exp Immunol. 149(1), 139-45 (2007); Sasaki et al., Mol Hum Reprod. 10(5), 347-53 (2004); Schober et al., Immunol Cell Biol. 90(10), 935-44 (2012); Jiang et al., J of Immunol. 192(11), 4949-56 (2014)), a strategy that exploits the X-linked inheritance of Foxp3 and random inactivation of this chromosome in female Foxp3^{DTR/WT} mice heterozygous for co-expression of the high affinity human diphtheria toxin (DT) receptor with Foxp3 to consistently achieve partial transient depletion of maternal Foxp3⁺ cells to pre-pregnancy levels was utilized (Rowe et al., Cell Host Microbe. 10(1), 54-64 (2011); Kim et al., Nat Immunol. 8(2), 191-7 (2007); Samstein et al., Cell. 150(1):29-38 (2012)). This analysis showed partial transient ablation of bulk maternal Foxp3⁺ Tregs triggers significantly increased CXCL9 expression by decidual neutrophil (CD11b⁺ Ly6C^{int}) and macrophage (F4/80⁺ CD11b⁻) cells, along with expanded accumulation of fetal-OVA-specific CD8⁺ T cells at the maternal-fetal interface (FIGS. 10A and 10B). These immune cell shifts in the decidua parallel sharply increased rates of fetal wastage among Foxp3^{DTR/WT} mice bearing allogeneic pregnancy administered DT (FIG. 10C) (Rowe et al., Cell Host Microbe. 10(1), 54-64 (2011); Rowe et al., Nature. 490(7418), 102-6 (2012)). To investigate the therapeutic benefits of CXCR3 blockade in this non-infectious context, fetal resorption and loss of live pups were compared among DT treated Foxp3^{DTR/WT} mice administered CXCR3 neutralizing antibody either prior to, or 12 or 24 hours after DT administration. It was found that CXCR3 neutralization initiated before or 12 hours after initiating DT treatment each conferred near complete protection against fetal wastage induced by partial depletion of maternal Foxp3⁺ Tregs (FIG. 10C). Conversely, the protective benefits on fetal resorption and loss of live pups were mitigated when CXCR3 blockade was delayed until 24 hours after DT administration (FIG. 10C). Thus, protection against fetal wastage conferred by CXCR3 neutralization initiated before or shortly after prenatal Lm infection extends to disruptions in fetal tolerance triggered by partial depletion of maternal Foxp3⁺ Tregs.

### Discussion

The maternal immune system is charged with two monumental responsibilities during pregnancy, avert rejection of the fetus bearing foreign paternal antigens while simultaneously maintaining immunity against pathogenic microbes (Rowe et al., Reproduction. 146(6), R191-203 (2013)). This is accomplished, in large part, by anatomically segregating antigenically discordant fetal tissue, and concentrating immune silencing mechanisms where they are needed most, at the maternal-fetal interface (Erlebacher et al., Annu Rev Immunol. 31 387-411 (2013)). In this regard, non-overlapping strategies that restrict maternal immune cell access to the decidua and suppress local activation of potentially harmful immune components have been identified. These include diminished and skewed MHC expression by trophoblast cells (Zuckermann et al., J Immunol. 137(3), 846-53 (1986); Hunt et al., J Immunol. 138(8), 2481-7 (1987)), impaired decidual complement activation and deposition (Xu et al., Science. 287(5452), 498-501 (2000)), tryptophan catabolism through indoleamine 2,3-dioxygenase (Munn et al., Science. 281(5380), 1191-3 (1998)), expanded immune suppressive maternal Foxp3+ Tregs (Rowe et al., Cell Host Microbe. 10(1):54-64 (2011); Aluvihare et al., Nat Immunol. 5(3), 266-71 (2004)), uterine entrapment of antigen presenting cells (Collins et al., J Clin Invest. 119(7), 2062-73 (2009)), along with decidual exclusion of IFN-γ producing Th1/Tc1 T cells through chemokine gene silencing (Nancy et al., Science. 336(6086), 1317-21 (2012)). However, an inevitable consequence of intensely focused immune silencing in this fashion is vulnerability to infection by pathogens capable of breaching this ‘immune privileged’ site (Robbins et al., Curr Opin Microbiol. 15(1), 36-43 (2012)). Therefore, it is not surprising microbes with tropism for infecting trophoblasts and other placental cells have widely been shown to cause pregnancy complications (Gibbs, Semin Perinatol. 26(1), 75-8 (2002); Goldenberg et al., Lancet. 375(9724), 1482-90 (2010); Robbins et al., Curr Opin Microbiol. 15(1), 36-43 (2012)).

Along with direct in utero pathogen invasion, recent findings suggest overriding normally silenced maternal immune components that prevent fetal rejection by prenatal infection also contribute to the immune pathogenesis of fetal wastage (Rowe et al., PLoS Pathog. 8(8), e1002873 (2012); Rowe et al., Reproduction. 146(6), R191-203 (2013)). For example after Lm prenatal infection, fetal resorption directly parallels systemic expansion and activation of maternal T cells with fetal specificity, despite infection parameters that preclude direct in utero fetal invasion (Rowe et al., PLoS Pathog. 8(8), e1002873 (2012)). Based on these observations, it is suggested that infection induced disruptions in fetal tolerance instigate fetal wastage by drawing maternal immune cells and circulating pathogen to the maternal-fetal interface (Rowe et al., PLoS Pathog. 8(8), e1002873 (2012); Rowe et al., Reproduction. 146(6), R191-203 (2013)). However, underlying questions regarding the specific maternal immune components responsible for fetal injury, their necessity for fetal antigen recognition and decidual infiltration in driving fetal wastage remained elusive.

These outstanding issues are reconciled with results described in this current study. In particular, the necessity for maternal CD8+ T cells with fetal specificity and their recruitment to the decidua in infection induced fetal wastage is definitively established using transgenic mouse tools that fix maternal T cell specificity, and complementary loss-of-function approaches for averting decidual infiltration by maternal T cells with fetal specificity. Consistent with previously described CXCL9 repression enforced through histone methylation and other epigenetic modifications among decidual stromal cells (Nancy et al., Science. 336(6086), 1317-21 (2012)), only background levels of CXCL9 expression were found among non-leukocytes isolated from the decidua after prenatal infection. Nonetheless, CXCL9 producing CD11b+Ly6Cint neutrophil and F4/80+ macrophage cells that infiltrate this tissue after Lm prenatal infection functionally circumvents chemokine expression silencing at the maternal fetal interface. In turn, maternal CD8+ T cells with fetal specificity selectively upregulate expression of the CXCL9-chemokine receptor, CXCR3, and are recruited to the decidua. For immunity to microbes such as Lm and viral pathogens (e.g. herpes simplex virus and LCMV) that each reside within the host cell intracellular compartment, these findings are consistent with recruitment of protective pathogen-specific T cells to non-gestational female reproductive tissue after exogenous CXCL9 administration or resident memory CD8+ T cell reactivation (Shin et al., Nature. 491(7424), 463-7 (2012); Schenkel et al., Nat. Immunol. 14(5), 509-13 (2013)). These results are also in agreement with prior descriptions of cellular infiltration, along with elevated expression of pro-inflammatory cytokines such as G-CSF, TNF-α, IL-6, IFN-γ and CXCL9 at the maternal-fetal interface after infection with other bacterial or parasitic pathogens (e.g. Salmonella, Plasmodium, Toxoplasma or Leishmania spp.) that cause vertical transmission (Chattopadhyay et al., Infect Immun. 78(5), 2292-301 (2010); Dong et al., Infect Immun. 80(9); 3034-8 (2012); Zhang et al., Am J Reprod Immunol. 67(2); 112-21 (2012); Krishnan et al., J Immunol. 156(2), 653-62 (1996); Avila-Garcia et al, Am J Tropical Med and Hygiene. 89(2), 354-8 (2013); Osorio et al., Am J Tropical Med and Hygiene. 86(5), 812-20 (2012); Eltoum et al., Am J Tropical Med and Hygiene 46(1); 57-62 (1992); Schloesser et al., Scandinavian Journal Infect Dis. 36(10), 773-4 (2004); Pejcic-Karapetrovic et al., J Immunol. 179(9), 6088-96 (2007)).

These findings further underscore the necessity for sustained immune tolerance systemically, and perhaps more importantly, at the maternal-fetal interface in maintaining pregnancy. In response to prenatal pathogens that cause disseminated infection and directly invade the decidua, systemic as well as local immune suppressive pathways are likely dampened as a means for more efficient pathogen eradication (Rowe et al., PLoS Pathog. 8(8), e1002873 (2012); Ertelt et al., J Immunol. 187(5), 2569-77 (2011); Rowe et al., Reproduction. 146(6), R191-203 (2013)). In turn, even partial disruptions in tolerance to immunologically discordant antigens expressed by the developing fetus unleash the activation and decidual recruitment of maternal effector T cells with fetal specificity. By contrast, prenatal infection with influenza A virus with distribution is shown to be confined to respiratory tissue with only isolated case reports of placental-fetal invasion (Irving et al., BJOG: Intl J Obstet Gynecol. 107(10), 1282-9 (2000); Ramphal et al., Am J Obstet Gynecol. 138(3), 347-8 (1980); Gu et al., Lancet. 370(9593), 1137-45 (2007); McGregor et al., Am J Obstet Gynecol. 149(8), 856-9 (1984); Yawn et al., JAMA. 216(6); 1022-3 (1971); Lieberman et al., Emerging Infect Dis. 17(1), 107-9 (2011); Dulyachai et al., Emerging Infect Dis. 16(2), 343-4 (2010)) does not cause fetal resorption after infection in mice bearing allogeneic pregnancy. Nonetheless, with regards to pathogens like Lm that cause systemic infection with tropism for decidual invasion, the present data demonstrates a necessity for Th1/Tc1 inducing chemokines in the pathogenesis of infection induced fetal wastage. Blockade of the Th1/Tc1 chemokine receptor, CXCR3, not only eliminates CD8+ T cell infiltration into the decidua, but also protects against in utero bacterial invasion and infection induced fetal resorption. Taken together, these results establish Th1/Tc1 T cell recruitment via CXCR3 not only promote, but are essential for the immune pathogenesis of fetal wastage and in utero fetal invasion after Lm prenatal infection.

By delaying CXCR3 neutralization after the initiation of Lm infection, sustained protective benefits that stem from disrupting this chemokine receptor pathway were also revealed. It was found that initiating CXCR3 blockade within the first 12 hours after prenatal infection conferred near complete protection against fetal resorption, loss of live pups, and in utero Lm fetal invasion comparable to CXCR3 neutralization prior to infection. Conversely, these beneficial outcomes were eliminated when CXCR3 blockade was delayed until 24 hours post-infection. It is encouraging that protection against Lm infection induced fetal wastage conferred by CXCR3 blockade occurred with similar kinetics and efficacy compared with, ampicillin, the clinical gold standard treatment of prenatal Lm infection (Silver, Obstet Gynecol Surv. 53(12), 737-40 (1998); Fleming et al., Obstet Gynecol. 66(3 Suppl), 52S-3S (1985); Kalstone, Am J Obstet Gynecol. 164(1 Pt 1), 57-8 (1991)). However, considering the inciting pathogen is undefined initially in most cases of prenatal infection, this more universal approach for reversing immune-mediated pregnancy complications may offer more comprehensive benefits compared with antimicrobial agents targeting individual pathogens.

Protection conferred by CXCR3 neutralization shown for pathogen-induced fetal wastage extends to mitigating fetal resorption and loss of live pups triggered by partial depletion of maternal immune suppressive Foxp3+ Tregs. Similar to prenatal Lm infection, partial transient ablation of maternal Tregs to pre-pregnancy levels primes expansion and IFN-γ production among fetal-specific maternal effector T cells (Rowe et al., Cell Host Microbe. 10(1), 54-64 (2011); Rowe et al., Nature. 490(7418), 102-6 (2012); Samstein et al., Cell. 150(1), 29-38 (2012)), illustrating that the sustained systemic expansion of this immune suppressive T cell subset is essential for maintaining fetal tolerance. Fetal wastage triggered by partial depletion of maternal Foxp3+ cells in mice directly parallel blunted expansion of maternal Tregs in the peripheral blood and decidua in human pregnancy complications associated with disruptions in fetal tolerance (e.g. preeclampsia, spontaneous abortion) (Prins et al., Hypertens Pregnancy. 28(3), 300-11 (2009); Santner-Nanan et al., J of Immunol. 183(11), 7023-30 (2009); Sasaki et al., Clin Exp Immunol. 149(1), 139-45 (2007); Sasaki et al., Mol Hum Reprod. 10(5), 347-53 (2004); Schober et al., Immunol Cell Biol. 90(10), 935-44 (2012); Jiang et al., J of Immunol. 192(11), 4949-56 (2014)), whereas overriding local immune suppressive pathways in place to sustain fetal tolerance likely plays decisive roles in the pathogenesis of fetal injury considering the absence of fetal wastage after infection with influenza A restricted from direct access to the maternal-fetal interface. Accordingly, this newfound pathway whereby decidual chemokine expression silencing becomes functionally circumvented may drive the underlying pathogenesis of fetal wastage after infectious as well as idiopathic disruptions in fetal tolerance possibility representing subclinical or undiagnosed local infection (Goldenberg et al., Am. J. of Obstetrics and Gynecology 198(3), 861-73 (2003); Gibbs, Semin. Perinatol. 26(1), 75-8 (2002); Goldenberg et al., Lancet, 375(9724), 1482-90 (2010)). Reciprocally, restoring repressed chemokine function at the maternal-fetal interface through CXCR3 neutralization may provide more universal protection against unintentional attack by maternal immune components with fetal specificity.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of reducing the risk of or preventing fetal wastage, the method comprising:
administering to a pregnant subject a CXCR3 inhibitor in an amount effective to reduce the risk of or prevent fetal wastage.

2. The method of claim 1, wherein the subject has or is at risk of having an infection, a stillbirth, preeclampsia or a premature infant.

3. The method of claim 2, wherein the subject has or is at risk of having an infection.

4. The method of claim 3, wherein the infection is caused by a pathogen selected from the group consisting of Listeria monocytogenes, Influenza A, herpes simplex virus, Lymphocytic Choriomeningitis Virus (LCMV), Salmonella, Plasmodium, Toxoplasma, Escherichia coli, CMV (cytomegalovirus), parvovirus, or Leishmania spp.

5. The method of claim 4, wherein the infection is caused by Listeria monocytogenes.

6. The method of claim 1, wherein the pregnant subject is a human female subject.

7. The method of claim 3, wherein the CXCR3 inhibitor is administered prior to the infection or prior to manifestation of a symptom of the infection.

8. The method of claim 3, wherein the CXCR3 inhibitor is administered to the subject within 7 days after the subject is infected with a pathogen or manifests a symptom of the infection.

9. The method of claim 1, wherein the CXCR3 inhibitor is selected from the group consisting of an interfering RNA, an antisense oligonucleotide, a small molecule, and an antibody.

10. The method of claim 9, wherein the CXCR3 inhibitor is an antibody that specifically binds to CXCR3.

11. The method of claim 10, wherein the CXCR3 is human CXCR3.

12. The method of claim 10, wherein the antibody is a human antibody or a humanized antibody.

13. The method of claim 1, wherein the amount of the CXCR3 inhibitor is effective to inhibit fetal-specific CD8+ T cells in the subject.

14. The method of claim 1, wherein the CXCR3 inhibitor is administered orally.

15.-29. (canceled)


SEQUENCE LISTING (4 sequences)

SEQ ID NO: 1 - DNA, 1670 bp, Homo sapiens
ccaaccacaagcaccaaagcagaggggcaggcagcacaccacccagcagccagagcaccagcccagccatggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctgacttccccgcattccaggctcctccctccctctgccggctctggctctccccaatatcctcgctcccgggactcactggcagccccagcaccaccaggtctcccgggaagccaccctcccagctctgaggactgcaccattgctgctccttagctgccaagccccatcctgccgcccgaggtggctgcctggagccccactgcccttctcatttggaaactaaaacttcatcttccccaagtgcggggagtacaaggcatggcgtagagggtgctgccccatgaagccacagcccaggcctccagctcagcagtgactgtggccatggtccccaagacctctatatttgctcttttatttttatgtctaaaatcctgcttaaaacttttcaataaacaagatcgtcaggaccaaaaaaaaaaaaaaaaaaaaaaaaaaaaaaaaaaaaaaaaaaaaaaa

SEQ ID NO: 2 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 3 - DNA, 1914 bp, Homo sapiens
ccaaccacaagcaccaaagcagaggggcaggcagcacaccacccagcagccagagcaccagcccagccatggtccttgaggggtccctgggccgatgggatcacgcagaagaatgcgagagaagcagcctttgagaagggaagtcactatcccagagcccaggctgagcggatggagttgaggaagtacggccctggaagactggcggggacagttataggaggagctgctcagagtaaatcacagactaaatcagactcaatcacaaaagagttcctgccaggcctttacacagccccttcctccccgttcccgccctcacaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctgacttccccgcattccaggctcctccctccctctgccggctctggctctccccaatatcctcgctcccgggactcactggcagccccagcaccaccaggtctcccgggaagccaccctcccagctctgaggactgcaccattgctgctccttagctgccaagccccatcctgccgcccgaggtggctgcctggagccccactgcccttctcatttggaaactaaaacttcatcttccccaagtgcggggagtacaaggcatggcgtagagggtgctgccccatgaagccacagcccaggcctccagctcagcagtgactgtggccatggtccccaagacctctatatttgctcttttatttttatgtctaaaatcctgcttaaaacttttcaataaacaagatcgtcaggaccaaaaaaaaaaaaaaaaaaaaaaaaaaaaaaaaaaaaaaaaaaaaaaa

SEQ ID NO: 4 - protein, 415 aa, Homo sapiens
MELRKYGPGRLAGTVIGGAAQSKSQTKSDSITKEFLPGLYTAPSSPFPPSQVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL